(19) United States
(12) Patent Application Publication
(10) Pub. No.: US 2023/0046904 A1
(43) Pub. Date: Feb. 16, 2023

(54) COMBINATION OF SMALL MOLECULE INHIBITOR OF THE PD-1/PD-L1 INTERACTION AND ANTI-PD-1 ANTIBODY FOR TREATING CANCER

(71) Applicant: GUANGZHOU MAXINOVEL PHARMACEUTICALS CO., LTD., Guangdong (CN)

(72) Inventors: Yuguang WANG, Shanghai (CN); Feilan WANG, Beijing (CN); Nong ZHANG, Shanghai (CN)

(21) Appl. No.: 17/761,097

(22) PCT Filed: Sep. 17, 2020
(86) PCT No.: PCT/CN2020/115743

§ 371 Date: Mar. 16, 2022

(30) Foreign Application Priority Data
Sep. 17, 2019 (CN) PCT/CN2019/106128
Sep. 11, 2020 (CN) 202010951925.X

(51) Int. Cl. A61K 31/198; A61K 39/395; A61K 45/06

(52) U.S. Cl. CPC A61K 31/198; A61K 39/3955; A61K 45/06; A61K 2039/545

(57) ABSTRACT

The invention provides methods for treating a cancer, comprising administering to a subject in need thereof a therapeutically effective amount of a small molecule inhibitor of the PD-1/PD-L1 interaction or a pharmaceutically acceptable salt or prodrug thereof in combination with a therapeutically effective amount of an anti-PD-1 antibody, wherein the small molecule inhibitor of the PD-1/PD-L1 interaction is not a protein.

This is a National Phase application filed under 35 US.C. 371 as a national stage of PCT/CN2020/115743, filed Sep. 17, 2020. an application claiming the benefit of Chinese Application No. 202010951925.X filed Sep. 11, 2020 and International Application No. PCT/CN2019/106128, filed Sep. 17, 2019. the content of each of which is hereby incorporated by reference in its entirety.

### FIELD OF INVENTION

This invention relates to methods for treating cancers using a combination of small molecule inhibitors targeting the interaction of PD-1 and PD-L1 and anti-PD-1 antibodies.

### PRIOR ARTS

PD-1 (Programmed death 1, CD279) is a member of the CD28 super family, which is mainly distributed in immune-related cells, such as T cells, B cells and NK cells. It plays an important role in immune response processes, e.g., autoimmune diseases, tumors, infections, organ transplantation or allergies.

Programmed death-1 (PD-1) is the major receptor for PD-L1. Programmed death-ligand 1 (PD-L1), also known as B7-H1, belongs to the B7 family and is widely distributed in peripheral tissues and hematopoietic cells. It is induced by various cytokines, e.g., IFN-γ, and it is expressed on T cells, NK cells, macrophages, myeloid DCs, B cells, epithelial cells, and vascular endothelial cells. PD-L1 is highly expressed in various tumors, such as lung cancer, gastric cancer, melanoma and breast cancer, and it is thought to help cancers to evade the host immune system.

PD-1/PD-L1 binding exerts a negative immuno modulatory effect. For example, when PD-1 on the surface of T cells interacts with PD-L1 on the surface of tumor cells or tumor-associated macrophages, the interaction causes a series of signaling responses leading to inhibition of T lymphocyte proliferation and secretion of related cytokines, apoptosis of tumor antigen-specific T cells, and/or incapable immunization, ultimately suppressing the immune response and allowing the escape of tumor cells. Monoclonal antibodies targeting PD-1 or PD-L1 can break the immune tolerance of tumors by specifically blocking the interaction of PD-1/PD-L1, restore the killing function of tumor-specific T cells on tumor cells, and achieve clearance of tumors. Up to now, there are four PD-1 antibody drugs and four PD-L1 antibody drugs in China and in the US. The approved PD-1 antibody drugs include Merck's Keytruda® (pembrolizumab), Bristol-Myers Squibb's Opdivo® (nivolumab), Junshi Bioscience's Toripalimab, Hengrui Medicine's Camrelizumab and Innovent's Sintilimab. The approved PD-L1 antibody drugs include Atezolizumab by Roche, Durvalumab® by AstraZeneca, Avelumab® by Pfizer and Merck (Germany), and Cemiplimab by Regeneron. In addition, a number of other companies are developing PD-1/PD-L1 targeted antibodies.

Many cancer patients benefit from monoclonal antibodies to PD-1/PD-L1. However, studies have found that anti-PD-1/PD-L1 antibodies are not effective in all cancer patients. Clinical trial data show the effective response rate of anti-PD-1/PD-L1 antibody alone is about 20%.

Small molecule inhibitors binding to PD-1/PD-L1 are also actively developed. WO2018006795 and WO2019128918, which are incorporated herein by reference in their entirety, disclose novel small molecule inhibitors targeting the interaction of PD-1 and PD-L1. The small molecule inhibitors disclosed therein exhibit an anti-tumor effect in a mouse tumor model.

There is a need to improve the effective response rate in cancer immunotherapy, particularly in the case of patients who do not respond to monoclonal antibodies to PD-1/PD-L1.

### SUMMARY OF THE INVENTION

The present invention is based upon the discovery that the combination of a small molecule inhibitor of the PD-1/PD-L1 interaction and an anti-PD-1 antibody is more effective in treating cancer than either drug alone.

In one aspect, the invention provides methods for treating a cancer, comprising administering to a subject in need thereof a therapeutically effective amount of a small molecule inhibitor of the PD-1/PD-L1 interaction or a pharmaceutically acceptable salt or prodrug thereof and a therapeutically effective amount of an anti-PD-1 antibody, wherein the small molecule inhibitor of the PD-1/PD-L1 interaction is not a protein. In some embodiments, the anti-PD-1 antibody is a monoclonal antibody. For example, the anti-PD-1 antibody may be pembrolizumab (Keytruda®), nivolumab (Opdivo®), cemiplimab (Libtay®), toripalimab, Camrelizumab or sintilimab. In some embodiments, the small molecule inhibitor of the PD-1/PD-L1 interaction has a molecular weight (MW) less than 1500 Daltons. In some embodiments, the small molecule inhibitor of the PD-1/PD-L1 interaction has an IC₅₀ of less than 100 nM in a PD-1/PD-L1 binding assay. In some embodiments, the small molecule inhibitor of the PD-1/PD-L1 interaction is an aromatic vinyl or aromatic ethyl derivative. In a preferred embodiment, the small molecule inhibitor of the PD-1/PD-L1 interaction is

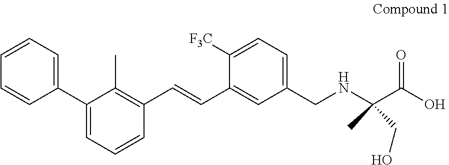 [SMILES: Cc1c(/C=C/c2cc(CN[C@@](C)(CO)C(=O)O)ccc2C(F)(F)F)cccc1-c1ccccc1]

in free or pharmaceutically acceptable salt form.

In some embodiments, the method further comprises administering to the subject an additional anti-cancer agent (e.g., other checkpoint inhibitors (e.g., anti CTLA-4 antibody) or chemotherapeutic agents).

In another aspect, the invention provides uses of a small molecule inhibitor of the PD-1/PD-L1 interaction or a pharmaceutically acceptable salt or prodrug thereof in the manufacture of a medicament for use in combination with an anti-PD-1 antibody in treating a cancer, wherein the small molecule inhibitor is not a protein.

### CONTENT OF THE PRESENT INVENTION

Unless specifically stated otherwise herein, references made in the singular may also include the plural. For example, “a” and “an” may refer to either one, or one or more.

Listed below are definitions of various terms used to describe the present disclosure. These definitions apply to the terms as they are used throughout the specification (unless they are otherwise limited in specific instances) either individually or as part of a larger group. The definitions set forth herein take precedence over definitions set forth in any patent, patent application, and/or patent application publication incorporated herein by reference.

As used herein, “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, an “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In some embodiments, the individual or subject is a human.

As used herein, “protein” means a compound consisting of at least 50 amino acids linked in a chain, the alpha-carboxyl group of each amino acid being joined to the alpha-amino group of the next by an amide bond, including protein multimers, e.g., antibodies, post-translationally modified proteins, e.g., glycosylated proteins, and proteins complexed with metals.

As used herein, “therapeutically effective amount” is intended to include an amount of a compound of the present disclosure alone or an amount of the combination of compounds claimed or an amount of a compound of the present disclosure in combination with other active ingredients effective to act as an inhibitor of the interaction of PD-1 and PD-L1, or effective to treat or prevent cancer.

As used herein, “treatment” or “treating” is an approach for obtaining beneficial or desired results including and preferably clinical results. For purposes of this invention, beneficial or desired clinical results include, but are not limited to, one or more of the following: decreasing symptoms resulting from the disease, increasing the quality of life of those suffering from the disease, decreasing the dose of other medications required to treat the disease, delaying the progression of the disease, and/or prolonging survival of individuals.

Reference to “about” a value or parameter herein includes (and describes) embodiments that are directed to that value or parameter per se. For example, description referring to “about X” includes description of “X.”

It is understood that embodiments, aspects and variations of the invention described herein include “consisting” and/or “consisting essentially of” embodiments, aspects and variations.

Cancer drug therapy has gone through several stages from chemotherapy, to targeted therapy, to immunotherapy during the past 50 years. While chemotherapy and targeted therapy involve in drugs to directly target cancer cells, immunotherapy relies on drugs to modulate the patient's own immune system which, in turns, kills the tumor cells. Thus, there are differences in therapeutic effects and toxicity profiles among the three therapies. Currently, immunotherapy is gaining the leadership role due to its durable response to some tumors and low occurrence of side effects.

The most successful immunotherapy is immuno checkpoint inhibition (ICI) therapy. Since the 2011 FDA approval of ipilimumab (anti-CTLA4) for the treatment of metastatic melanoma, more immuno checkpoint inhibitors, all targeting the PD-1/PD-L1 pathway, have been approved for the treatment of a broad range of tumor types. ICI targets inhibitory ligand-receptor interactions between T cells and immunosuppressive cells within the tumor microenvironment (TME), in particular, interactions mediated by tumor cells (Pardon, D. M. Nat. Rev. Cancer 2012, 12, 252-264). Malignant tumors often co-opt immune suppressive and tolerance mechanisms to avoid immune destruction. Anti-PD-1 or anti-PD-L1 antibodies inhibit T cell-negative costimulation to unleash antitumor T-cell responses that recognize tumor antigens.

PD-1, expressed upon activation of T and B lymphocytes, regulates T-cell activation through interaction with PD-L1 and PD-L2. (Wei, S. C. et al Cancer Discov. 2018, 8(9), 1069-86). When binding with PD-L1, PD-1 primarily transmits a negative costimulatory signal through the tyrosine phosphatase SHP2 to attenuate T-cell activation. Therefore, the inhibition of PD-1/PD-L1 pathway with anti-PD-1/L1 antibodies stops the negative costimulatory signal and restores the T-cell activation to achieve tumor inhibition.

Extensive studies of the commercially available PD-1/L1 antibody drugs have revealed how these antibody drugs interact with their target protein. The binding structures of anti-PD-1 antibody Pembrolizumab with PD-1 protein have been disclosed (Tan, S. et al Protein Cell 2016, 7:866-877). Crystal structures of Pembrolizumab fragment complexed with hPD-1 showed the molecular basis of therapeutic antibody-based immuno checkpoint inhibition of tumors. The interaction of Pembrolizumab with hPD-1 is mainly located on two regions: the flexible C′D loop and the C, C′ strands.

The protein binding model of anti-PD-L1 antibody drugs such as Durvalumab has also been published in Tan, S. et al Protein Cell 2017. The molecular basis of Durvalumab-based PD-1/PD-L1 blockade is that the unbiased binding of Durvalumab VH and VL to PD-L1 provides steric clash to abrogate the binding of PD-1/PD-L1. This is quite different from anti-PD-1 antibody Pembrolizumab with its residues participating in competitive binding to the ligand.

These pieces of binding information of anti-PD1/L1 antibody drugs at molecular level provide critical starting point for us to design the next generation immuno checkpoint inhibitors. Since the current antibody ICI therapy works for only 20-30% of the patients, it is in great need to develop the next generation drugs as soon as possible. Therefore, the next generation immuno checkpoint inhibitors require: 1) wider treatment response to more tumors than the current antibody therapy; 2) patient friendly oral dosing regimen; 3) effective brain penetration, and 4) shorter half-life for side effect management.

In the present invention, it has been found that that the combination of a small molecule inhibitor of the PD-1/PD-L1 interaction and an anti-PD-1 antibody is more effective in inhibiting tumor growth than either drug alone. Thus, the present invention relates to the combination of a small molecule inhibitor of the PD-1/PD-L1 interaction and an anti-PD-1 antibody for treating cancer. The use of a small molecule inhibitor of the PD-1/PD-L1 interaction in combination with an anti-PD-1 antibody may provide an improved immuno therapeutcial efficacy to patients, especially patients who are not responsive to the anti-PD-1 antibody therapy alone. Furthermore, one concern with the immunotherapy using anti-PD-1 antibodies is that they can allow the immune system to attack some normal organs in the body, which can lead to several side effects in some patients. The use of a reduced dose of anti-PD-1 antibodies in combination with a small molecule inhibitor of the PD-1/PD-L1 interaction may be useful in avoiding or reducing side effects caused by anti-PD-1 antibodies.

In one aspect, the invention provides a method (Method 1) for treating a cancer, comprising administering to a subject in need thereof a therapeutically effective amount of a small molecule inhibitor of the PD-1/PD-L1 interaction or a pharmaceutically acceptable salt or prodrug thereof and a therapeutically effective amount of an anti-PD-1 antibody, wherein the small molecule inhibitor of the PD-1/PD-L1 interaction is not a protein, for an example,

1.1 Method 1, wherein the small molecule inhibitor binds to PD-L1.

1.2 Method 1 or 1.1, wherein the small molecule inhibitor is an aromatic vinyl or aromatic ethyl derivative.

1.3 Any foregoing method, wherein the small molecule inhibitor has a molecular weight of less than 1500 Daltons.

1.4 Any foregoing method, wherein the small molecule inhibitor has an IC₅₀ less than 100 nM in a PD-1/PD-L1 binding assay, e.g., an assay as described in WO2018006795.

1.5 Method 1, wherein the small molecule inhibitor is an aromatic acetylene or aromatic ethylene PD-L1 inhibitor, e.g., as described in WO2018006795, incorporated herein by reference.

1.6 Method 1, wherein the small molecule inhibitor is an aromatic ethylene or aromatic ethyl PD-L1 inhibitor, e.g., as described in WO2019128918, incorporated herein by reference.

1.7 Method 1, wherein the small molecule inhibitor is a benzyl phenyl ether PD-L1 inhibitor, e.g., as described in WO2015034820 and/or WO2015160641, the contents of which applications are incorporated herein by reference, for example, BMS-1001 or BMS-1166.

1.8 Method 1, wherein the small molecule inhibitor is a derivative of aromatic ethylene or aromatic ethyl of formula (I), or a pharmaceutically acceptable salt, a metabolite, a metabolic precursor or a prodrug thereof;

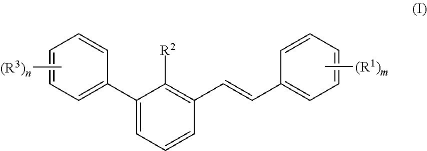

wherein,

[symbol] is a single bond or a double bond;

each of R¹ is the same or different, is independently selected from deuterium, halogen, a substituted or unsubstituted hydroxyl, a substituted or unsubstituted amino, a substituted or unsubstituted alkyl, or a substituted or unsubstituted alkoxyl; or the two adjacent R¹(s) together with the carbon atoms on the phenyl to which they are attached to form a 5- to 7-membered carboncyclyl or heterocyclyl together; the heterocyclyl is a heterocyclyl wherein the heteroatom is selected from the group consisting of oxygen and/or nitrogen, the number of the heteroatom(s) is 1 to 4;

R² is selected from a substituted or unsubstituted alkyl or a halogen;

each of R³ is the same or different, is independently selected from deuterium, halogen, a substituted or unsubstituted alkylthio, a substituted or unsubstituted hydroxyl, a substituted or unsubstituted amino, a substituted or unsubstituted alkyl, a substituted or unsubstituted alkoxyl,

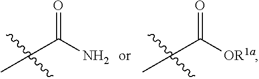

wherein R^{1a} is C₁₋₄ alkyl, or the two adjacent R³(s) together with the carbon atoms on the phenyl to which they are attached to form a 5- to 7-membered carbocyclyl or heterocyclyl together; the heterocyclyl is a heterocyclyl wherein the heteroatom is selected from the group consisting of oxygen and/or nitrogen, the number of the heteroatom(s) is 1 to 4; when two R³(s) are adjacent, and two R³(s) and two carbon atoms connected with them to form a 5- to 7-membered carboncyclyl or heterocyclyl together, the carboncyclyl or heterocyclyl is further substituted by one or more C₁₋₄ alkyl;

the substituted alkyl in each of R¹, R² and R³, the substituted alkoxyl in each of R¹, and R³ and the substituted alkylthio in each of R³, is selected one or more from the group consisting of halogen, C₁₋₄ alkyl, hydroxyl,

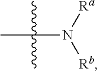

C₁₋₄ alkoxyl, C₁₋₄ carboxyl, C₁₋₄ ester group and C₁₋₄ amide group; when there are many substituents, the substituents are the same or different; R^{a} and R^{b} are independently selected from halogen, or, a substituted or unsubstituted alkyl; R^{a} and R^{b} can also be independently selected from hydrogen, or, a substituted or unsubstituted alkyl; in R^{a} or R^{b}, the substituents of a substituted alkyl are selected from the group consisting of halogen, C₁₋₄ alkyl, hydroxyl,

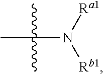

C₁₋₄ alkoxyl, C₁₋₄ carboxyl, C₁₋₄ ester group and C₁₋₄ amide group; R^{a1} and R^{b1} are independently selected from hydrogen or C₁₋₄ alkyl;

in each of R¹ or R³, the substituents of a substituted hydroxyl or a substituted amino are selected one or more from the group consisting of C₁₋₄ alkyl, C₁₋₄ alkoxyl, C₁₋₄ carboxyl, C₁₋₄ ester group and C₁₋₄ amide group;

m is 1, 2, or 3;

n is 0, 1, 2 or 3;

when [symbol] is a double bond, m is 2, and the two R¹(s) are occupied in ortho and meta positions of the phenyl, respectively, the two R¹(s) are the same or different;

when [symbol] is a double bond, m is 3, the two R¹(s) are adjacent, and the two adjacent R¹(s) together with the carbon atoms on the phenyl to which they are attached to form a 5- to 7-membered heterocyclyl together; or the derivative of aromatic ethylene or aromatic ethyl group of formula (I),

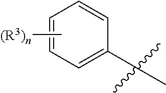

is replaced by a substituted or unsubstituted hetero aromatic ring, the heteroatom of the hetero aromatic ring is selected from oxygen, nitrogen or sulfur, the number of heteroatoms is 1-4; the substituents of a substituted hetero aromatic ring are selected one or more from the group consisting of halogen, C₁₋₄ alkyl, hydroxyl,

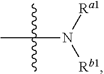

C₁₋₄ alkoxyl, C₁₋₄ carboxyl, C₁₋₄ ester group and C₁₋₄ amide group; the substituents of a substituted hetero aromatic ring can also be selected one or more from the group consisting of halogen, C₁₋₄ alkyl, hydroxyl,

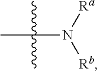

C₁₋₄ alkoxyl, C₁₋₄ carboxyl, C₁₋₄ ester group or C₁₋₄ amide group; when there are many substituents, the substituents are the same or different; R^{a1} and R^{b1} are independently selected from halogen, or, a substituted or unsubstituted alkyl; R^{a} and R^{b} can also be independently selected from hydrogen, or, a substituted or unsubstituted alkyl; in R^{a} or R^{b}, the substituents of a substituted alkyl are selected one or more from the group consisting of halogen, C₁₋₄ alkyl, hydroxyl,

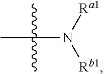

C₁₋₄ alkoxyl, C₁₋₄ carboxyl, C₁₋₄ ester group and C₁₋₄ amide group; R^{a1} and R^{b1} are independently selected from hydrogen or C₁₋₄ alkyl; and

the derivative of aromatic ethylene or aromatic ethyl group of formula (I) does not contain the following compounds:

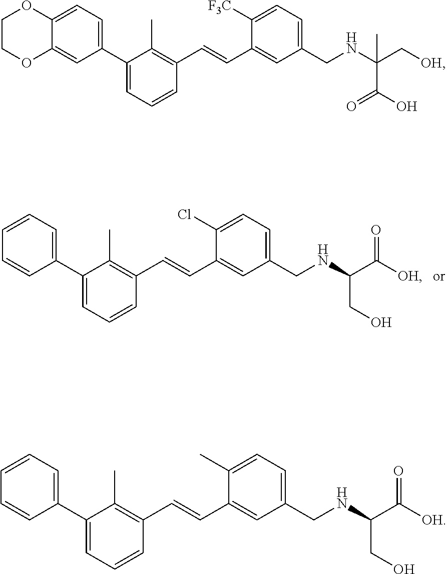 [SMILES: Cc1c(/C=C/c2cc(CNC(C)(CO)C(=O)O)ccc2C(F)(F)F)cccc1-c1ccc2c(c1)OCCO2 ; Cc1c(/C=C/c2cc(CN[C@H](CO)C(=O)O)ccc2Cl)cccc1-c1ccccc1 ; Cc1ccc(CN[C@H](CO)C(=O)O)cc1/C=C/c1cccc(-c2ccccc2)c1C]

1.9 Method 1, wherein the small molecule inhibitor is an aromatic acetylene or aromatic vinyl compound of the general formula II, in free or pharmaceutically acceptable salt form:

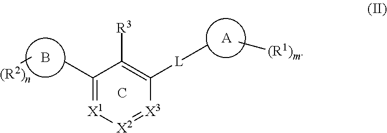

wherein, ring A and ring B are independently an aromatic ring or a heteroaromatic ring;

L is alkynyl, —C(R⁴)═C(R⁵)— or C₂₋₁₀ heteroaryl having at least one N;

X¹ is N or —CR⁶;

X² is N or —CR⁷;

X³ is N or —CR⁸;

X¹, X² and X³ are not N simultaneously;

each of R¹ is independently hydrogen, deuterium, substituted or unsubstituted hydroxy, substituted or unsubstituted amino, halogen, substituted or unsubstituted alkyl or substituted or unsubstituted alkoxy;

each of R² is independently hydrogen, deuterium, substituted or unsubstituted hydroxy, substituted or unsubstituted amino, halogen, substituted or unsubstituted alkyl or substituted or unsubstituted alkoxy,

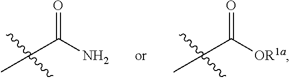

wherein R^{1a} is C₁₋₄ alkyl; or two adjacent R² together with the two atoms on the ring B to which they are attached form a 5-7 membered substituted or unsubstituted carbocycle, or substituted or unsubstituted heterocycle; in the heterocycle, heteroatom is oxygen and/or nitrogen, the number of the heteroatom(s) is 1-4;

R³ is deuterium, halogen, cyano, or substituted or unsubstituted alkyl;

R⁴ and R⁵ are each independently hydrogen, deuterium, substituted or unsubstituted alkyl, substituted or unsubstituted cycloalkyl, or R⁴ and R⁵ together with the carbon-carbon double bond to which they are attached form a 5-7 membered substituted or unsubstituted carbocycle, substituted or unsubstituted heterocycle; in the heterocycle, heteroatom is oxygen and/or nitrogen, the number of the heteroatom(s) is 1-4;

R⁶, R⁷ and R⁸ are each independently hydrogen, deuterium, substituted or unsubstituted hydroxy, halogen, substituted or unsubstituted amino, substituted or unsubstituted alkyl, or substituted or unsubstituted alkoxy, or R⁶ and R⁷ together with the two atoms on the ring C to which they are attached form a 5-7 membered substituted or unsubstituted heterocycle; or R⁷ and R⁸ together with the two atoms on the ring C to which they are attached form a 5-7 membered substituted or unsubstituted heterocycle, in the heterocycle, heteroatom is oxygen and/or nitrogen, the number of the heteroatom(s) is 1-4;

m is 1, 2 or 3;

n is 1 or 2;

in the definition of each R¹, the substituent in the substituted alkyl or the substituted alkoxy is selected from the group consisting of halogen, C₁₋₄ alkyl, hydroxy,

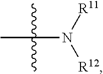

benzyl, benzyl substituted by cyano, C₁₋₄ alkoxy, C₁₋₄ carboxyl, C₁₋₄ ester group and C₁₋₄ acylamino; the substituent in the substituted hydroxy or the substituted amino is selected from the group consisting of C₁₋₄ alkyl, benzyl, benzyl substituted by cyano, C₁₋₄ alkoxy, C₁₋₄ carboxyl, C₁₋₄ ester group and C₁₋₄ acylamino;

in the definition of each R², the substituent in the substituted alkyl or the substituted alkoxy is selected from the group consisting of halogen, C₁₋₄ alkyl, hydroxy,

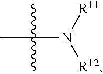

C₁₋₄ alkoxy, C₁₋₄ carboxyl, C₁₋₄ ester group and C₁₋₄ acylamino; the substituent in the substituted hydroxy or the substituted amino is selected from the group consisting of C₁₋₄ alkyl, benzyl, benzyl substituted by cyano, C₁₋₄ alkoxy, C₁₋₄ carboxyl, C₁₋₄ ester group and C₁₋₄ acylamino; when two adjacent R² together with the two atoms on the ring B to which they are attached form a 5-7 membered substituted carbocycle or substituted heterocycle, the substituent in the substituted carbocycle or in the substituted heterocycle is selected from the group consisting of halogen, C₁₋₄ alkyl, hydroxy,

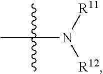

C₁₋₄ alkoxy, C₁₋₄ carboxyl, C₁₋₄ ester group and C₁₋₄ acylamino; when there are more substituents than one, the substituents are the same or different;

in the definition of R⁴ or R⁵, the substituent in the substituted alkyl or the substituted cycloalkyl is selected from the group consisting of halogen, C₁₋₄ alkyl, hydroxy, amino, C₁₋₄ alkoxy, C₁₋₄ carboxyl, C₁₋₄ ester group and C₁₋₄ acylamino; where R⁴ and R⁵ together with the carbon-carbon double bond to which they are attached form a 5-7 membered substituted carbocycle, or, substituted heterocycle, the substituent in the substituted carbocycle or the substituted heterocycle is selected from the group consisting of halogen, C₁₋₄ alkyl, hydroxy,

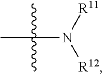

C₁₋₄ carboxyl, C₁₋₄ ester group and C₁₋₄ acylamino; when there are more substituents than one, the substituents are the same or different;

in the definition of R⁶, R⁷ or R⁸, the substituent in the substituted alkyl or the substituted alkoxy is selected from the group consisting of halogen, C₁₋₄ alkyl, hydroxy,

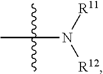

C₁₋₄ alkoxy, C₁₋₄ carboxyl, C₁₋₄ ester group and C₁₋₄ acylamino; the substituent in the substituted hydroxy or the substituted amino is selected from the group consisting of C₁₋₄ alkyl, benzyl, benzyl substituted by cyano, C₁₋₄alkoxy, C₁₋₄ carboxyl, C₁₋₄ ester group and C₁₋₄ acylamino; wherein R⁶ and R⁷ together with the two atoms on the ring C to which they are attached form a 5-7 membered substituted heterocycle, or when R⁷ and R⁸ together with the two atoms on the ring C to which they are attached form a 5-7 membered substituted heterocycle, the substituent in the substituted heterocycle is selected from the group consisting of halogen, C₁₋₄ alkyl, hydroxy,

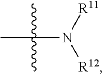

C₁₋₄ alkoxy, C₁₋₄ ester group and C₁₋₄ acylamino; when there are more substituents than one, the substituents are the same or different;

in

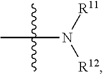

R¹¹ and R¹² are independently hydrogen, substituted or unsubstituted alkyl, alkoxy, hydroxyalkyl, aminoalkyl, substituted or unsubstituted C₆₋₁₄ aryl or substituted or unsubstituted C₃₋₆ cycloalkyl; or R¹¹ and R¹² together with the nitrogen atom to which they are attached form a 5-7 membered substituted or unsubstituted heterocycle; in the heterocycle, the heteroatom is nitrogen, or nitrogen and oxygen, the number of the heteroatom(s) is 1-4;

in the definition of R¹¹ and R¹², the substituent in the substituted alkyl, the substituted C₆₋₁₄ aryl or the substituted C₃₋₆ cycloalkyl is selected from the group consisting of halogen, C₁₋₄ alkyl, hydroxy,

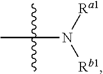

C₁₋₄ alkoxy, C₁₋₄ carboxyl, C₁₋₄ ester group and C₁₋₄ acylamino; when R¹¹ and R¹² together with the nitrogen atom to which they are attached form a 5-7 membered substituted or unsubstituted heterocycle, the substituent in the substituted heterocycle is selected from the group consisting of halogen, C₁₋₄ alkyl, substituted C₁₋₄ alkyl, hydroxy,

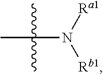

C₁₋₄ alkoxy, C₁₋₄ carboxyl, C₁₋₄ ester group and C₁₋₄ acylamino; the substituent in the substituted C₁₋₄ alkyl is selected from the group consisting of halogen, C₁₋₄ alkyl, hydroxy,

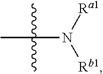

C₁₋₄ alkoxy, C₁₋₄ carboxyl, C₁₋₄ ester group and C₁₋₄ acylamino; when they are more substituents than one, the substituents are the same or different; in

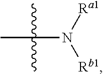

R^{a1} and R^{b1} are independently hydrogen, C₁₋₄ alkyl or

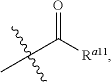

R^{a11} is C₁₋₄ alkyl.

1.10 Method 1, wherein the small molecule inhibitor is selected from a group consisting of:

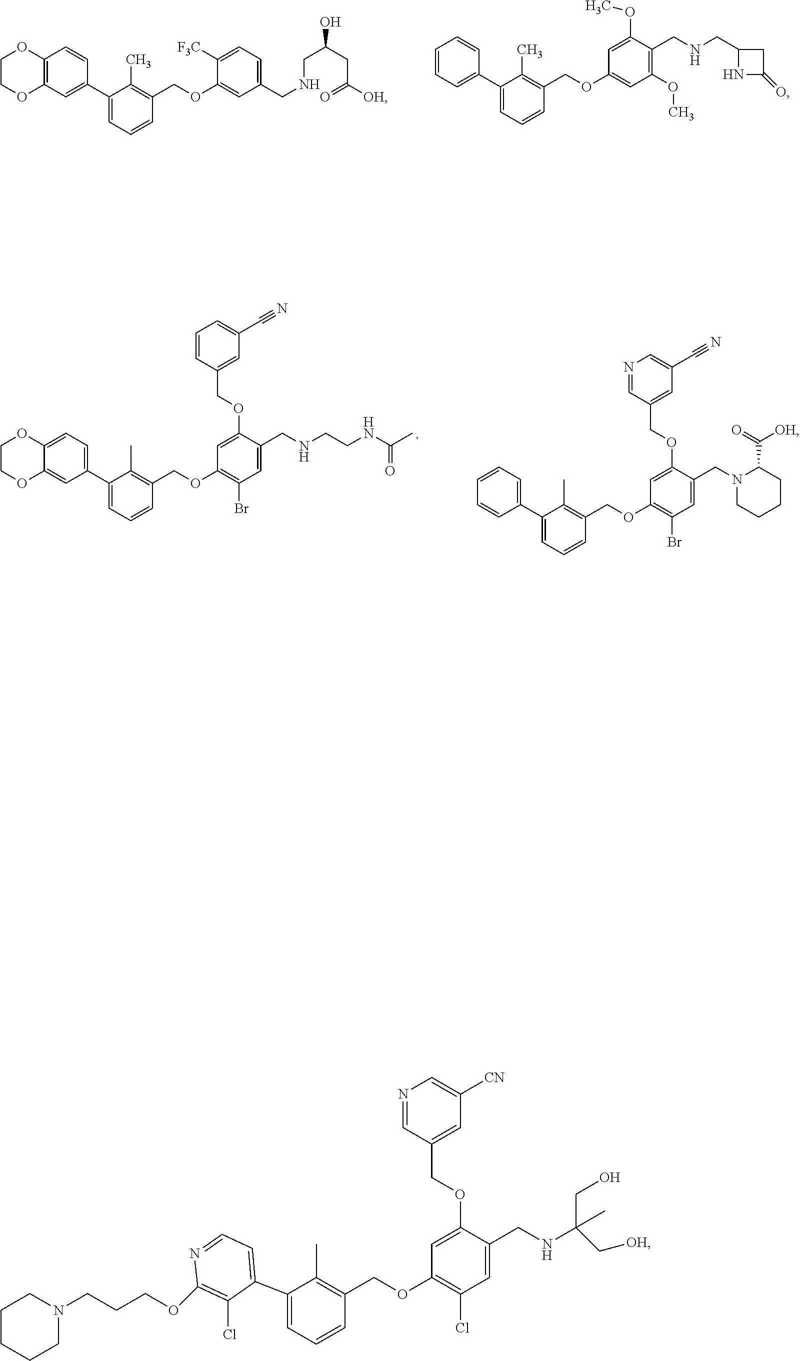 [SMILES: Cc1c(COc2cc(CNC[C@@H](O)CC(=O)O)ccc2C(F)(F)F)cccc1-c1ccc2c(c1)OCCO2 ; COc1cc(OCc2cccc(-c3ccccc3)c2C)cc(OC)c1CNCC1CC(=O)N1 ; CC(=O)NCCNCc1cc(Br)c(OCc2cccc(-c3ccc4c(c3)OCCO4)c2C)cc1OCc1cccc(C#N)c1 ; Cc1c(COc2cc(OCc3cncc(C#N)c3)c(CN3CCCC[C@H]3C(=O)O)cc2Br)cccc1-c1ccccc1 ; Cc1c(COc2cc(OCc3cncc(C#N)c3)c(CNC(C)(CO)CO)cc2Cl)cccc1-c1ccnc(OCCCN2CCCCC2)c1Cl]

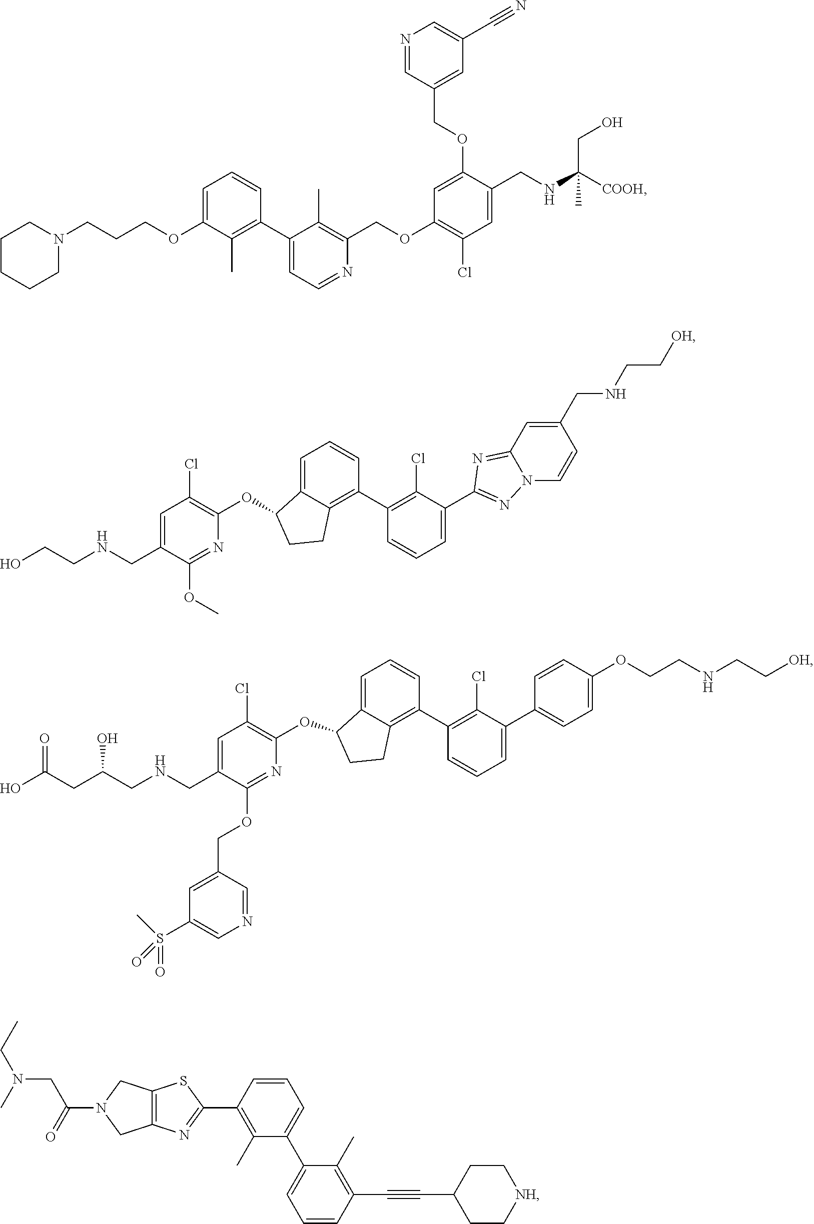 [SMILES: Cc1c(OCCCN2CCCCC2)cccc1-c1ccnc(COc2cc(OCc3cncc(C#N)c3)c(CN[C@@](C)(CO)C(=O)O)cc2Cl)c1C ; COc1nc(O[C@H]2CCc3c(-c4cccc(-c5nc6cc(CNCCO)ccn6n5)c4Cl)cccc32)c(Cl)cc1CNCCO ; CS(=O)(=O)c1cncc(COc2nc(O[C@H]3CCc4c(-c5cccc(-c6ccc(OCCNCCO)cc6)c5Cl)cccc43)c(Cl)cc2CNC[C@@H](O)CC(=O)O)c1 ; CCN(C)CC(=O)N1Cc2nc(-c3cccc(-c4cccc(C#CC5CCNCC5)c4C)c3C)sc2C1]

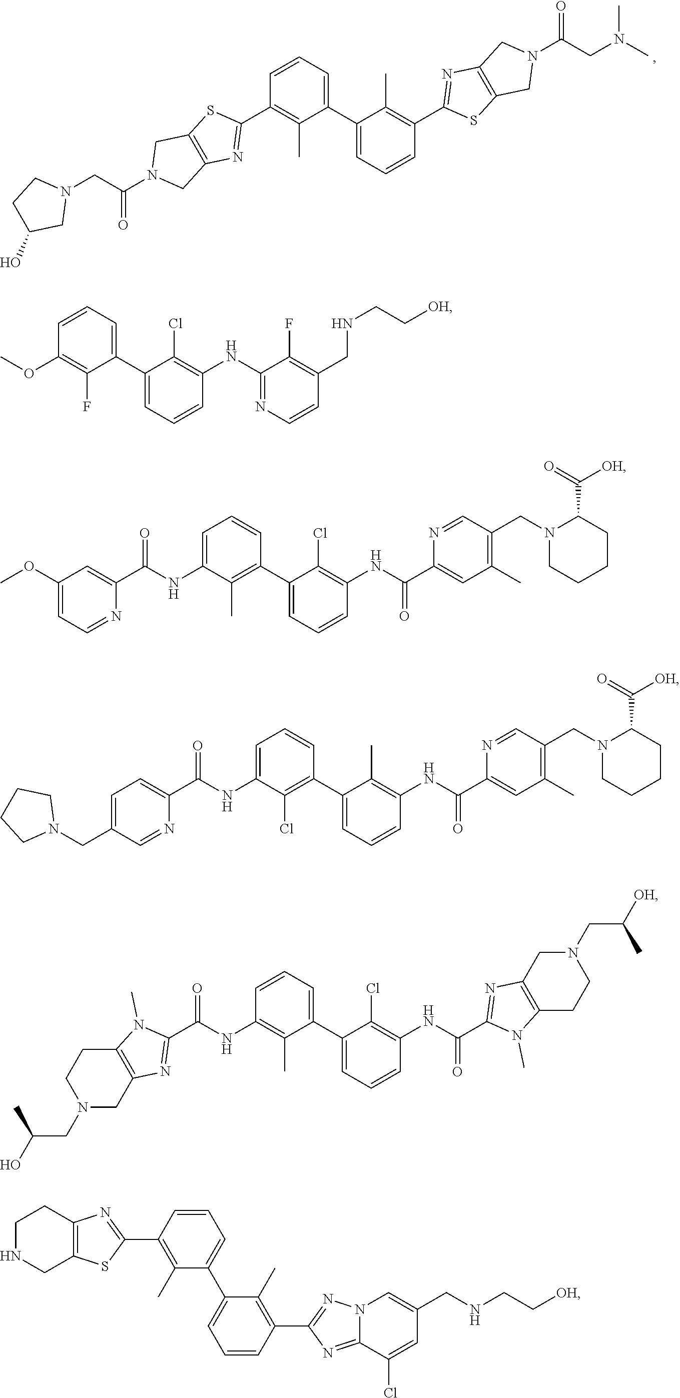

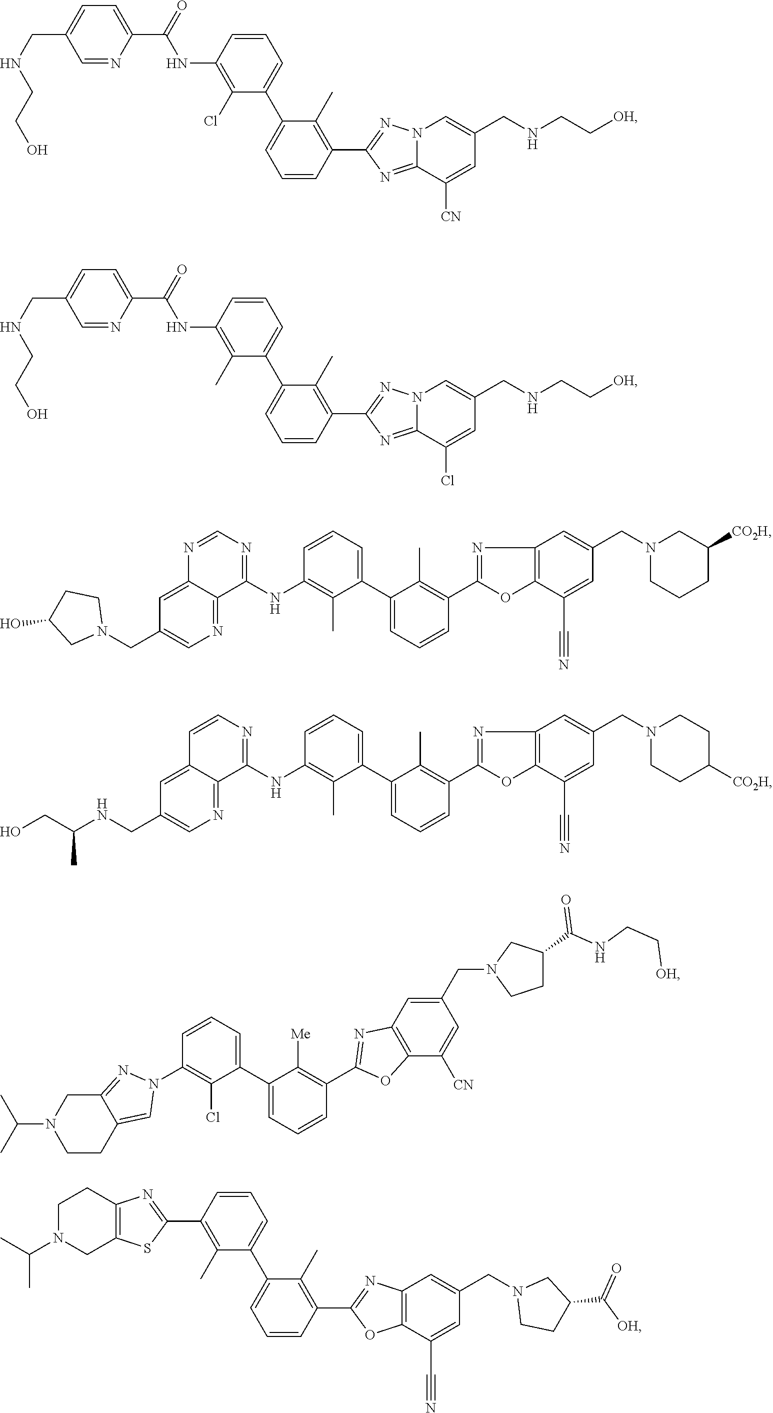

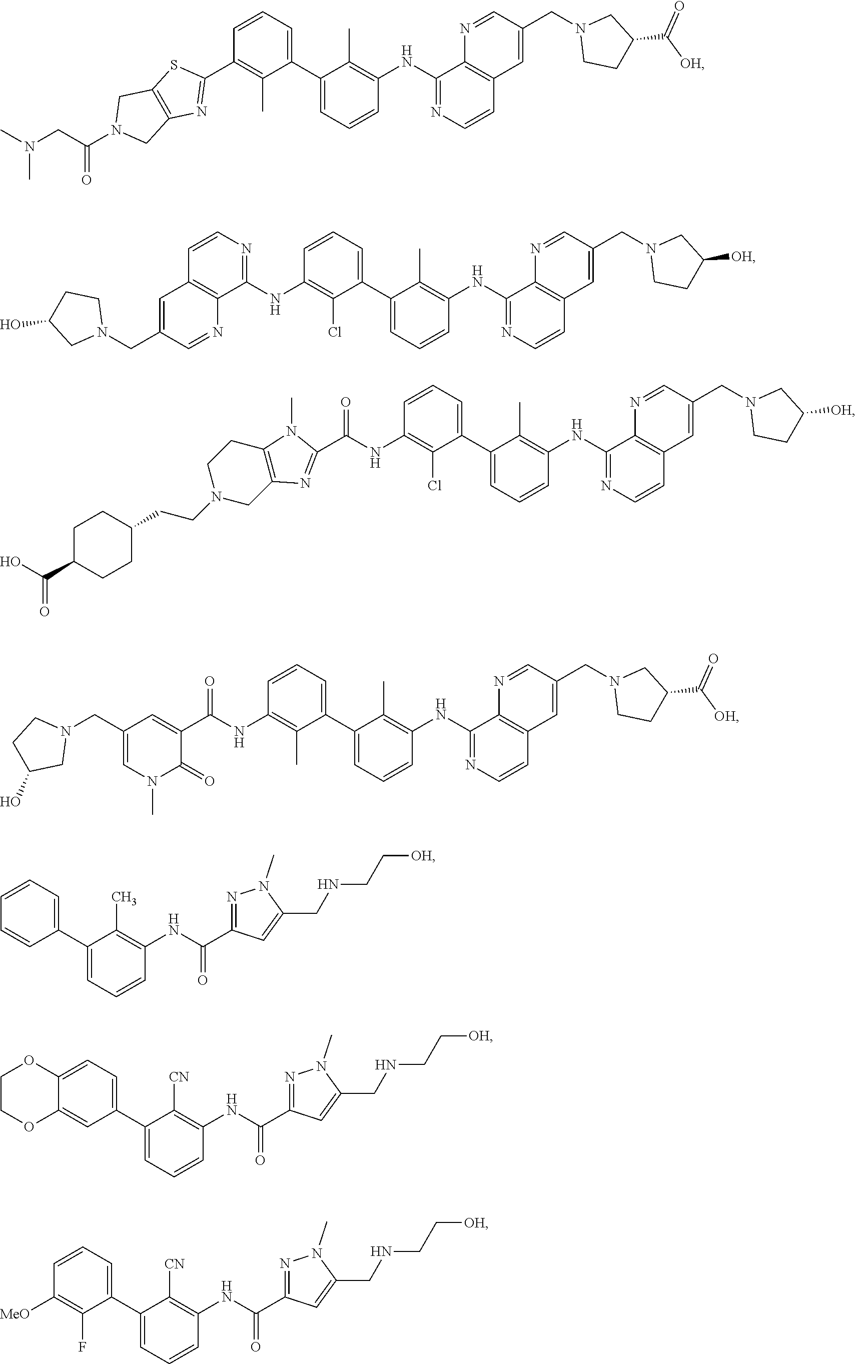

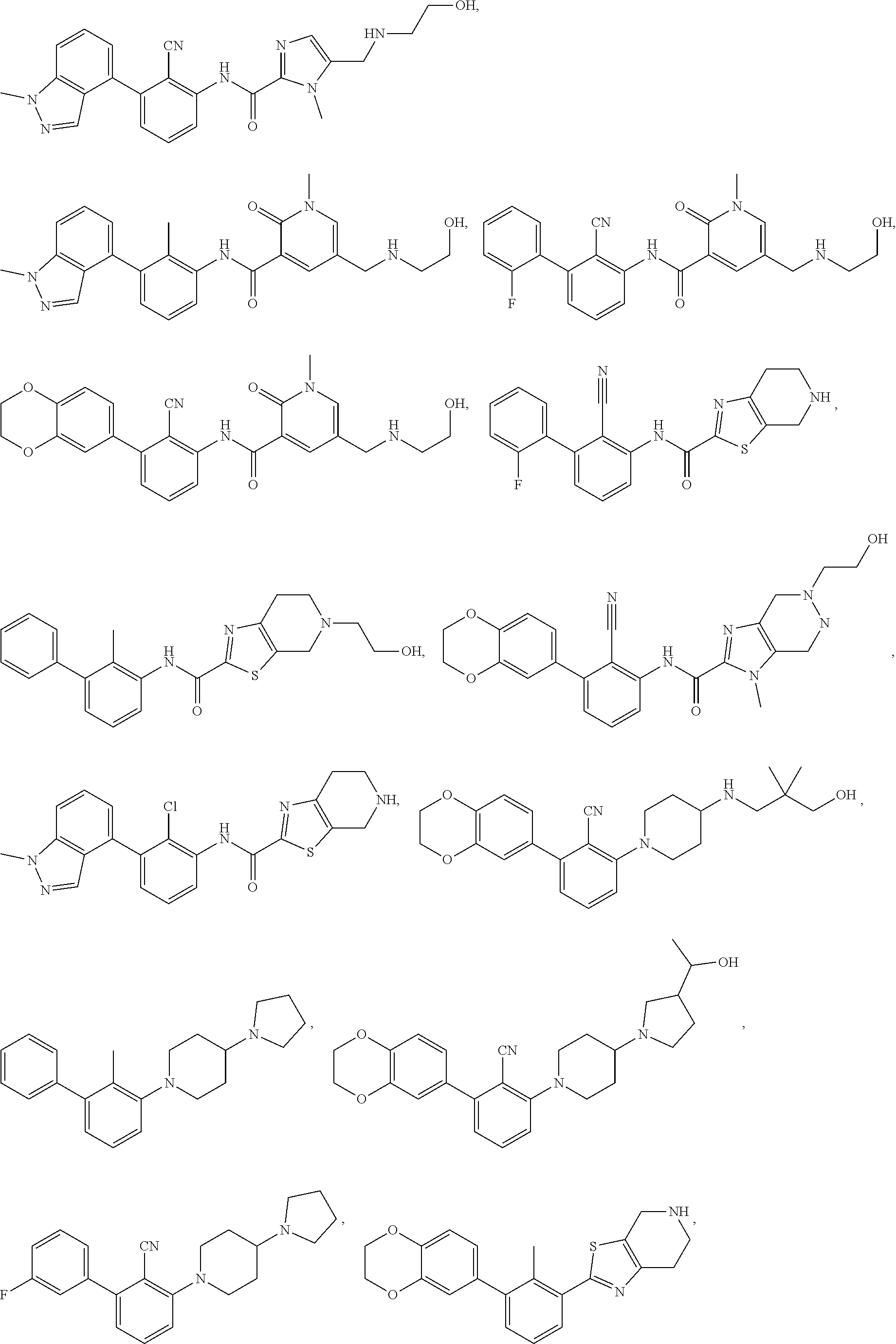

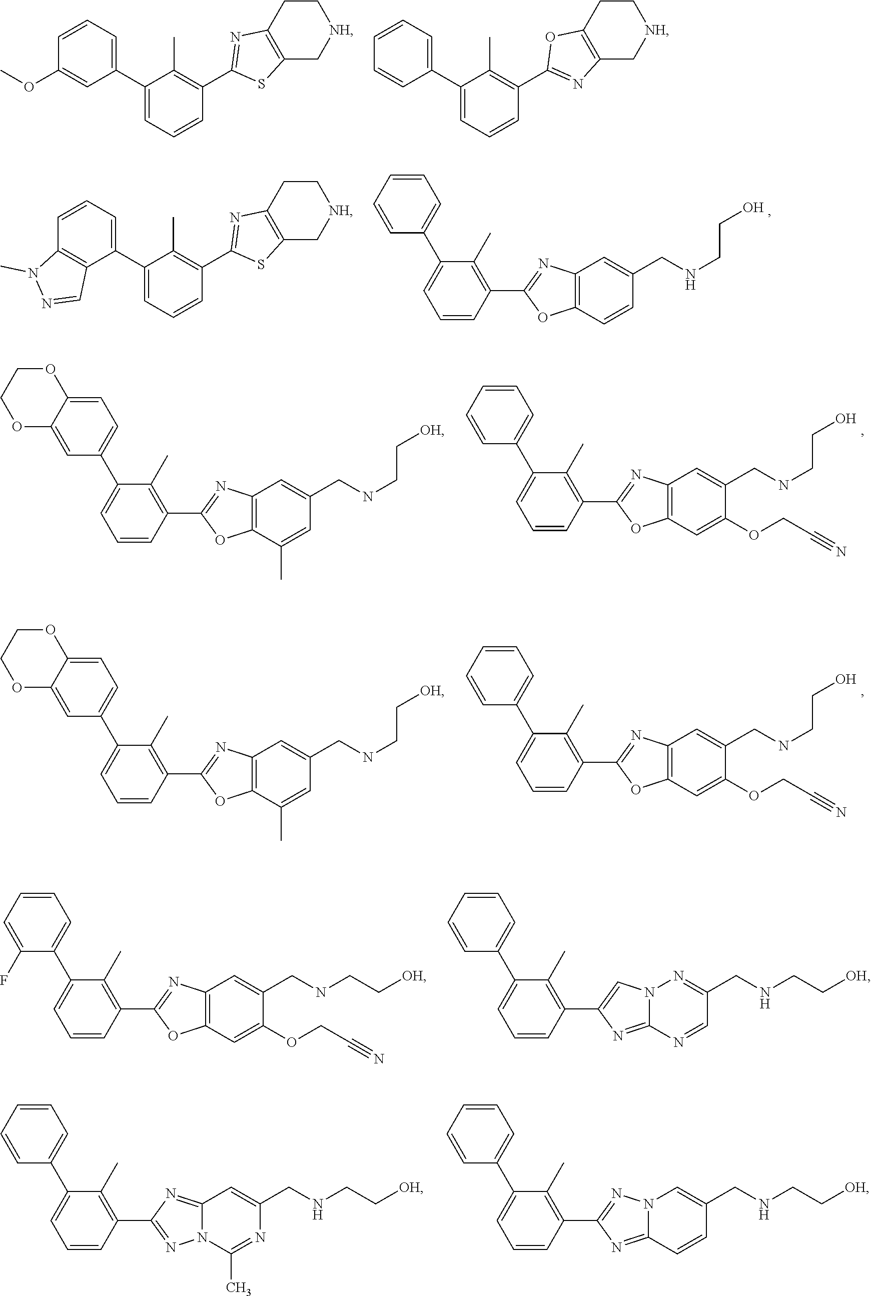

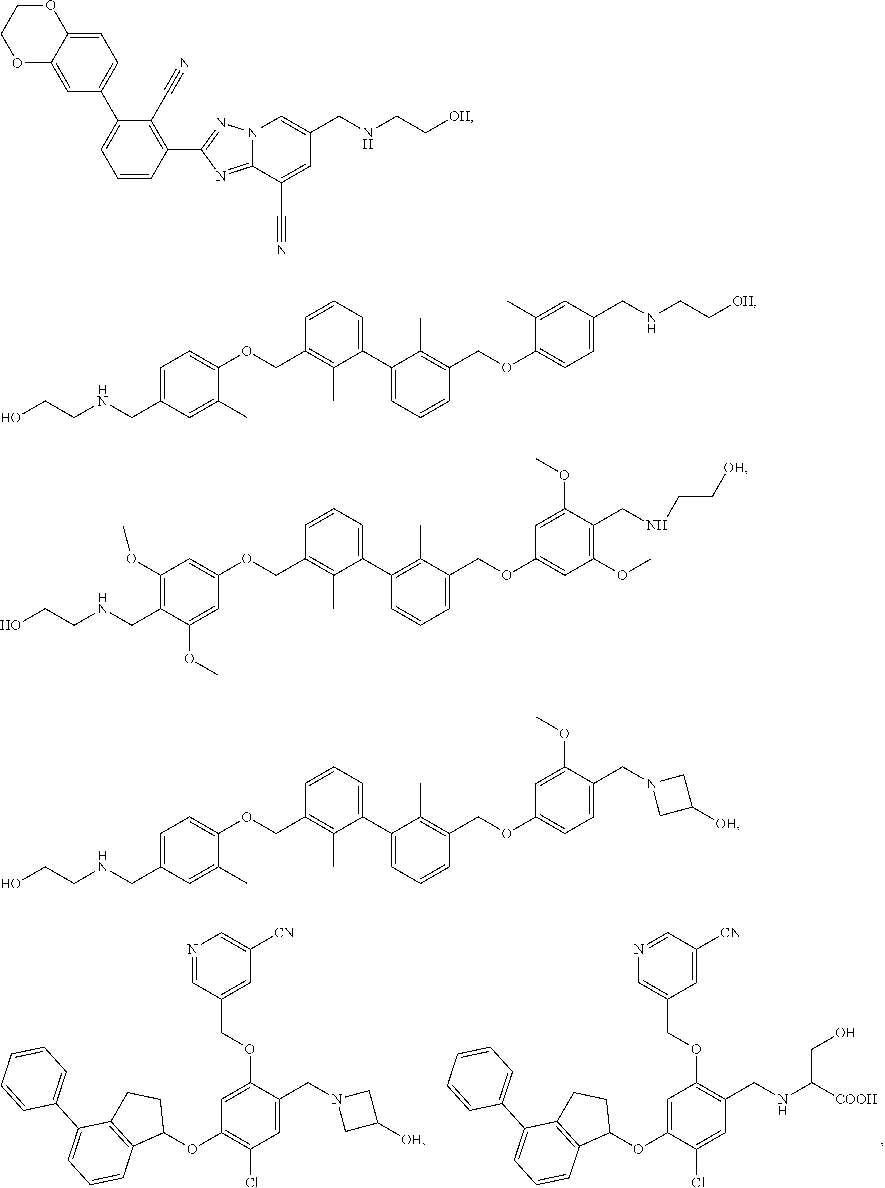 [SMILES: N#Cc1c(-c2ccc3c(c2)OCCO3)cccc1-c1nc2c(C#N)cc(CNCCO)cn2n1 ; Cc1cc(CNCCO)ccc1OCc1cccc(-c2cccc(COc3ccc(CNCCO)cc3C)c2C)c1C ; COc1cc(OCc2cccc(-c3cccc(COc4cc(OC)c(CNCCO)c(OC)c4)c3C)c2C)cc(OC)c1CNCCO ; COc1cc(OCc2cccc(-c3cccc(COc4ccc(CNCCO)cc4C)c3C)c2C)ccc1CN1CC(O)C1 ; N#Cc1cncc(COc2cc(OC3CCc4c(-c5ccccc5)cccc43)c(Cl)cc2CN2CC(O)C2)c1 ; N#Cc1cncc(COc2cc(OC3CCc4c(-c5ccccc5)cccc43)c(Cl)cc2CNC(CO)C(=O)O)c1]

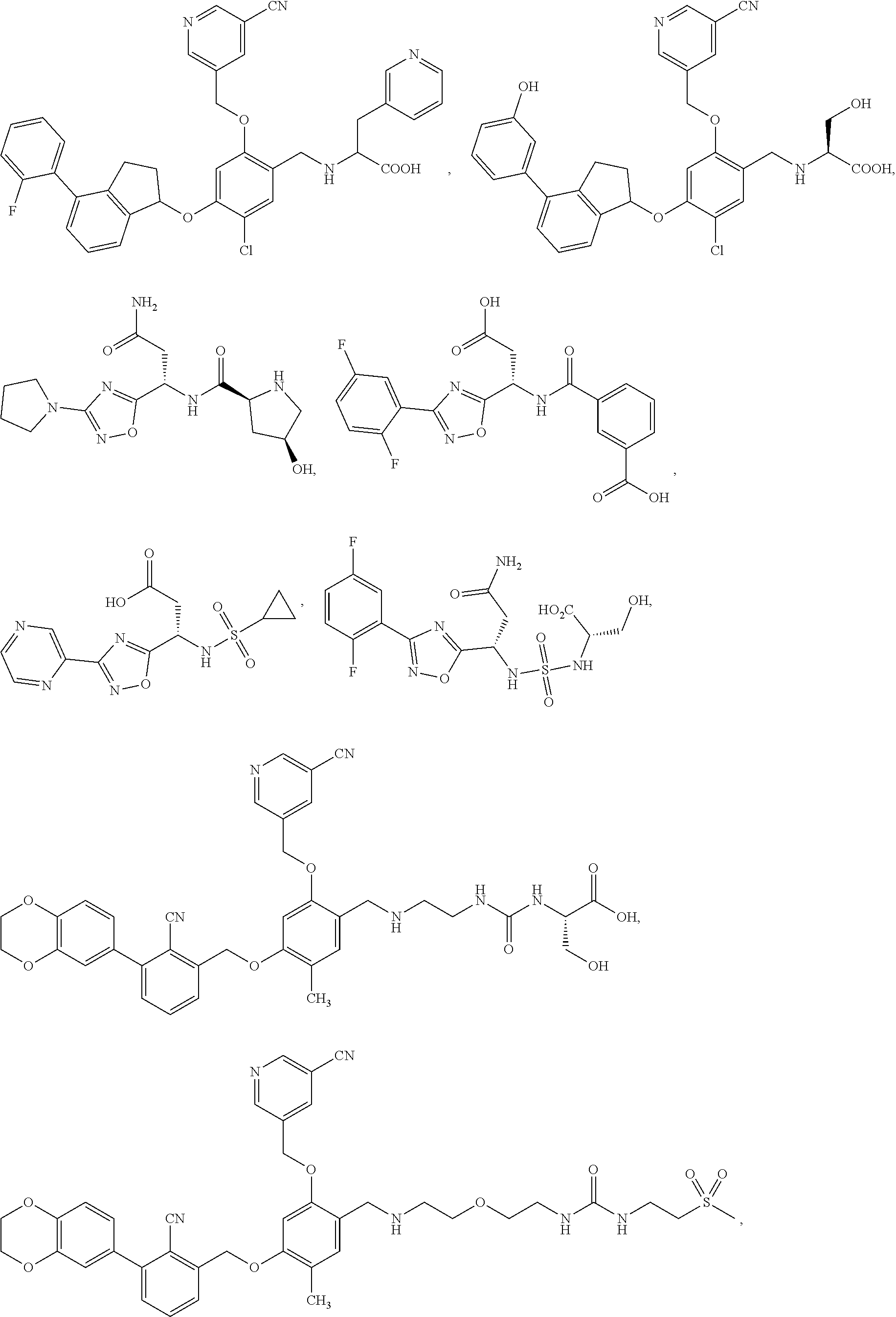

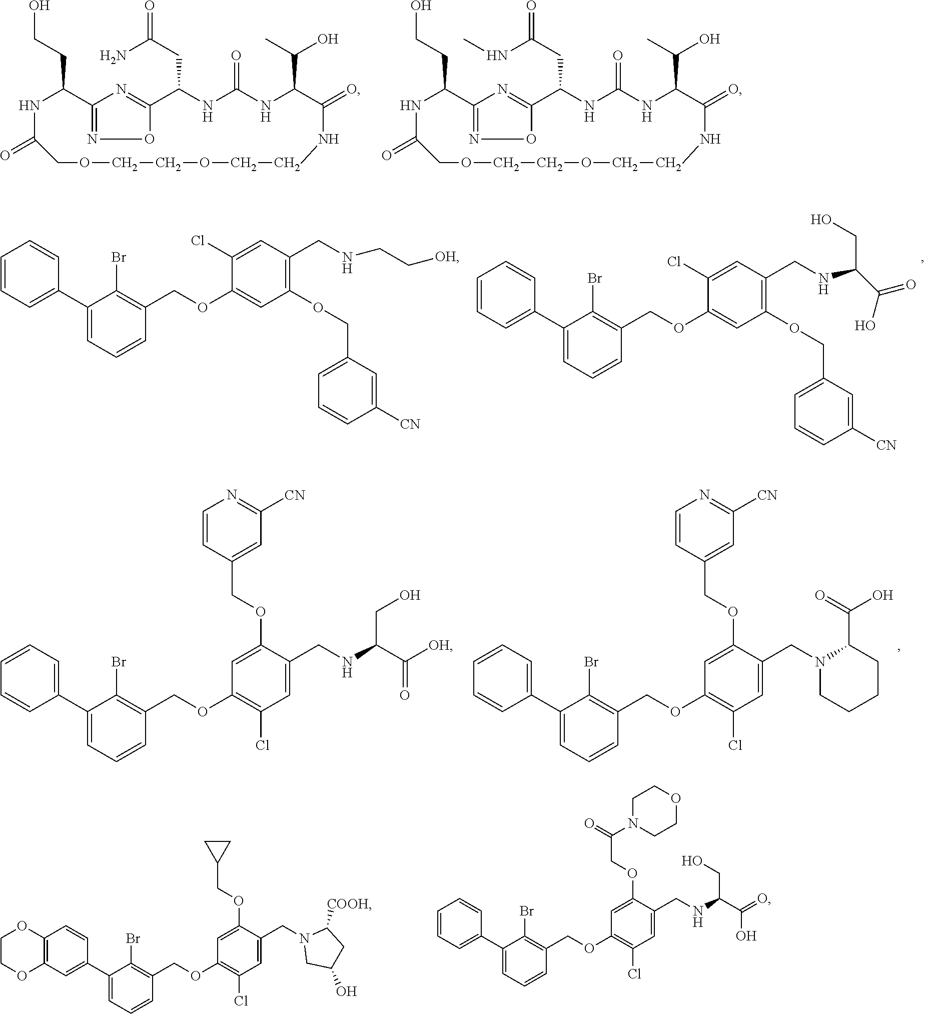

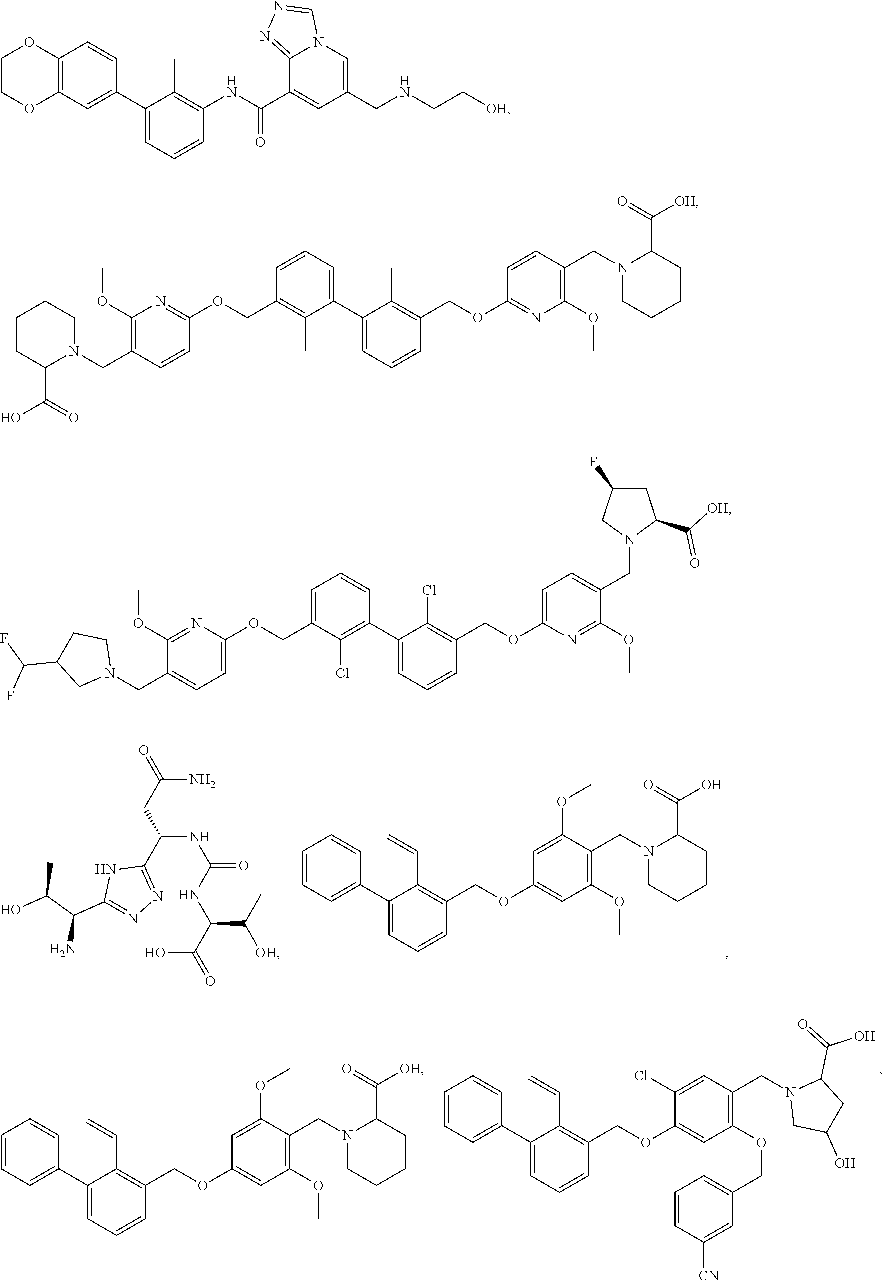

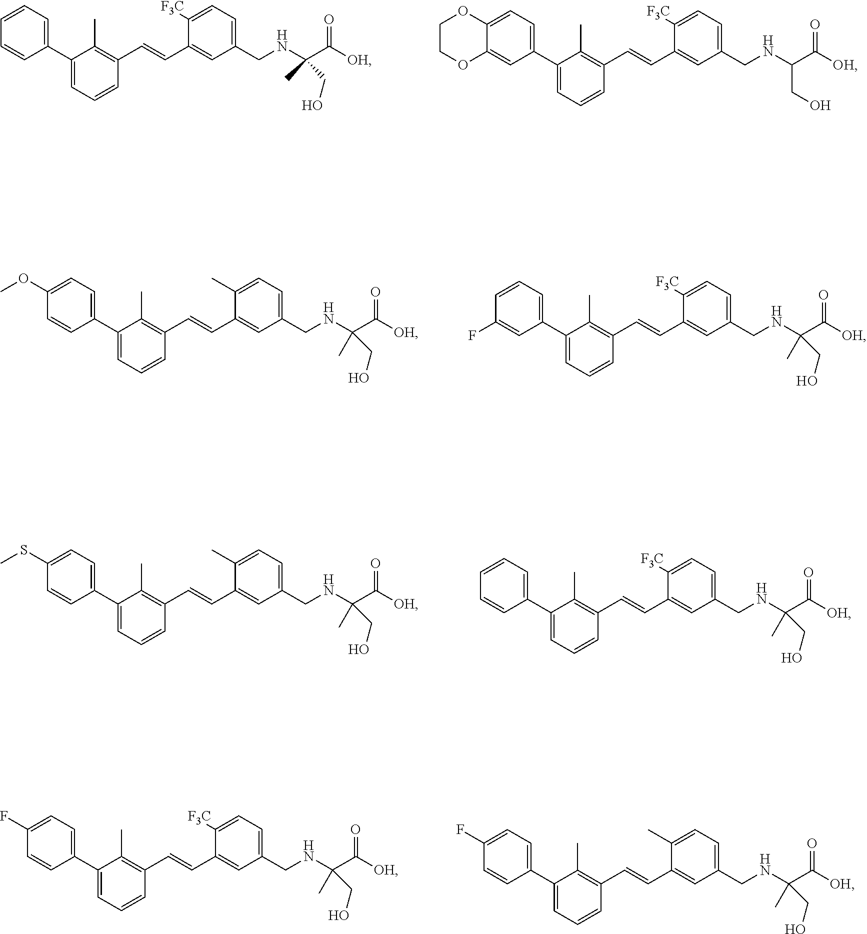

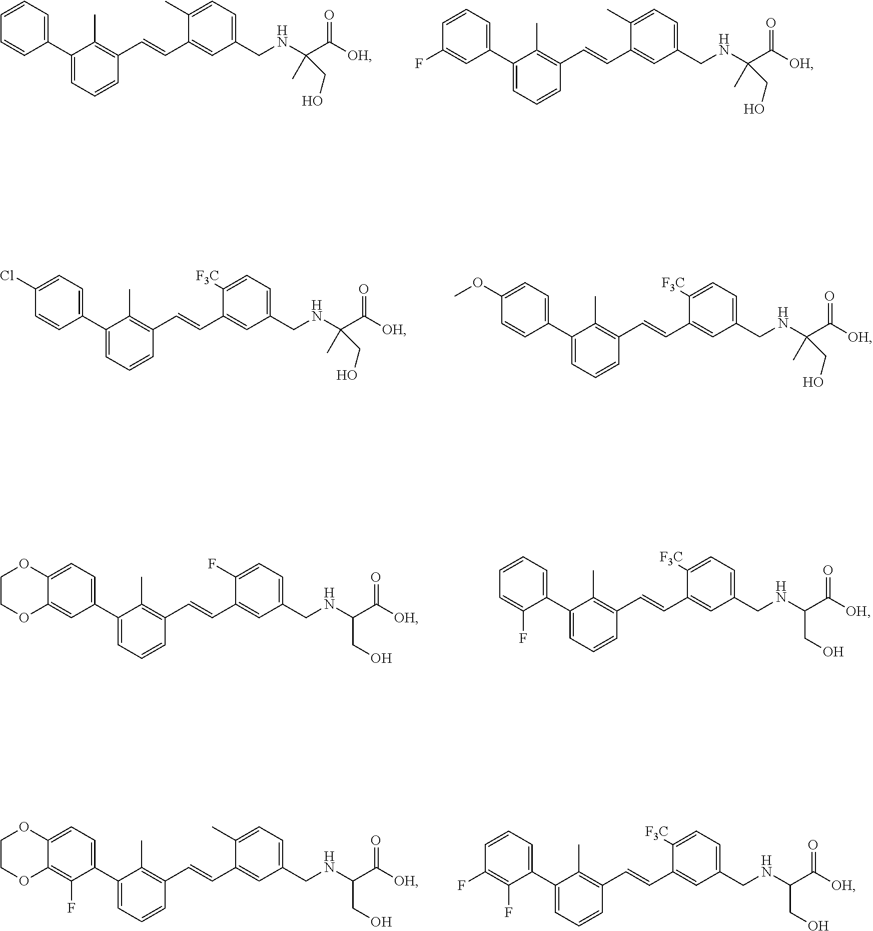

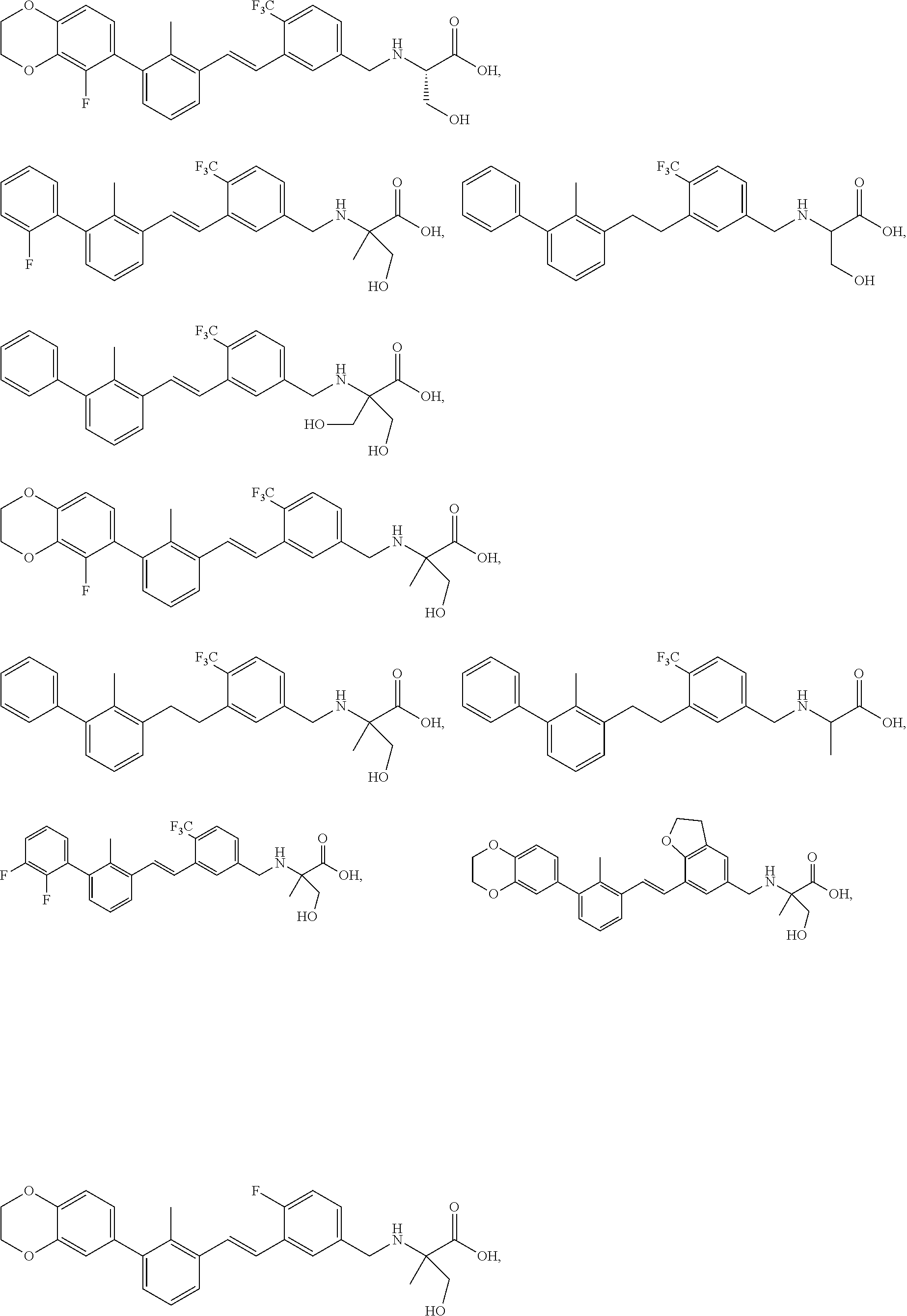

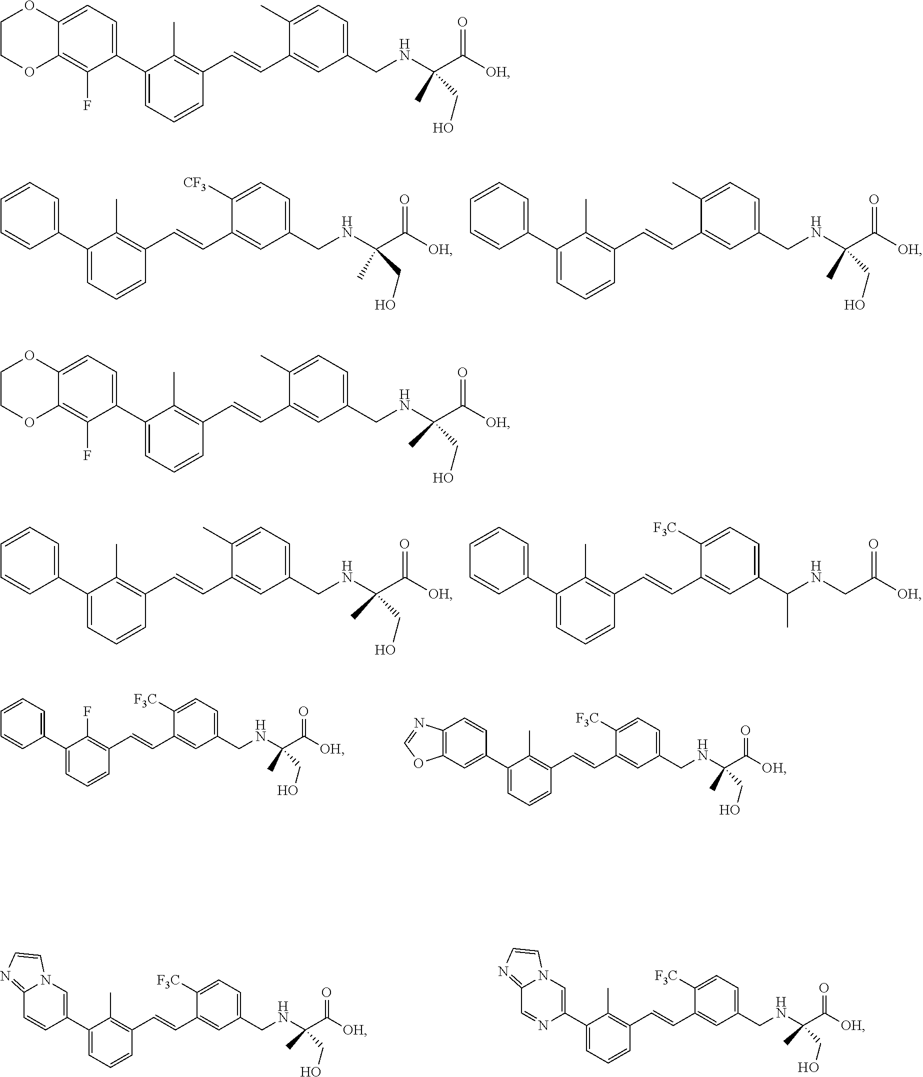

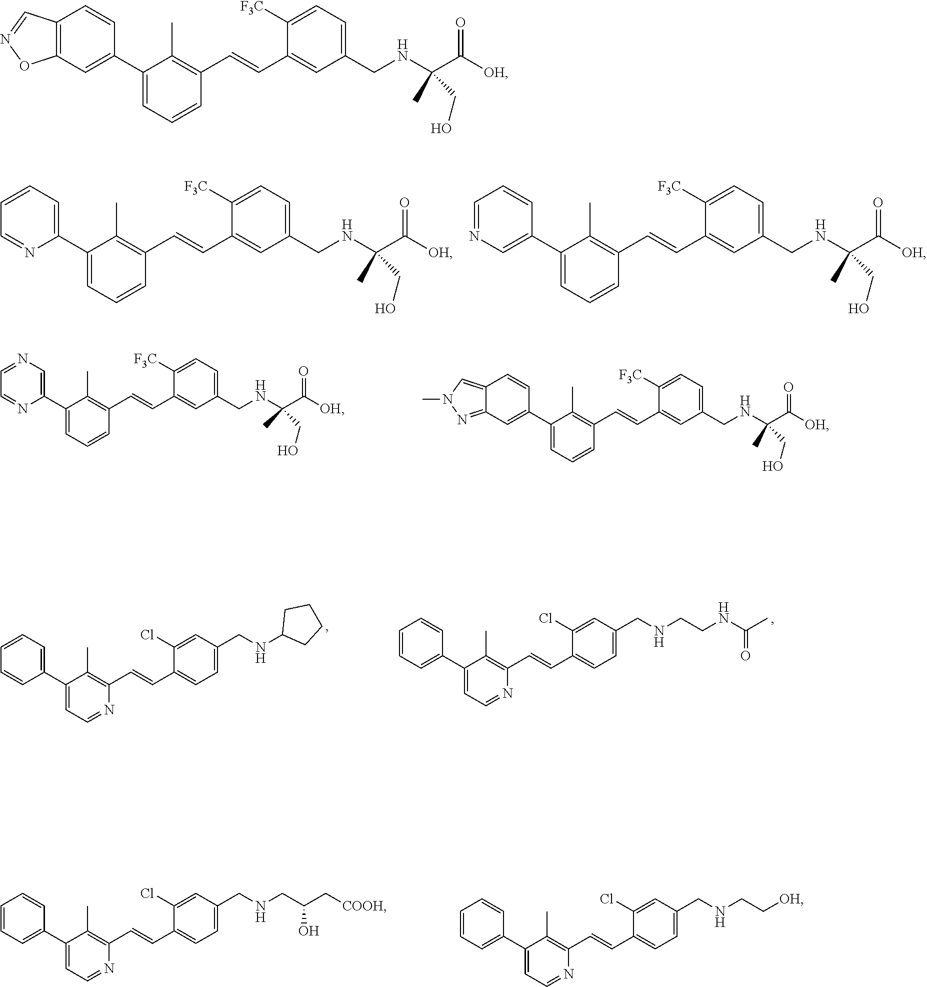

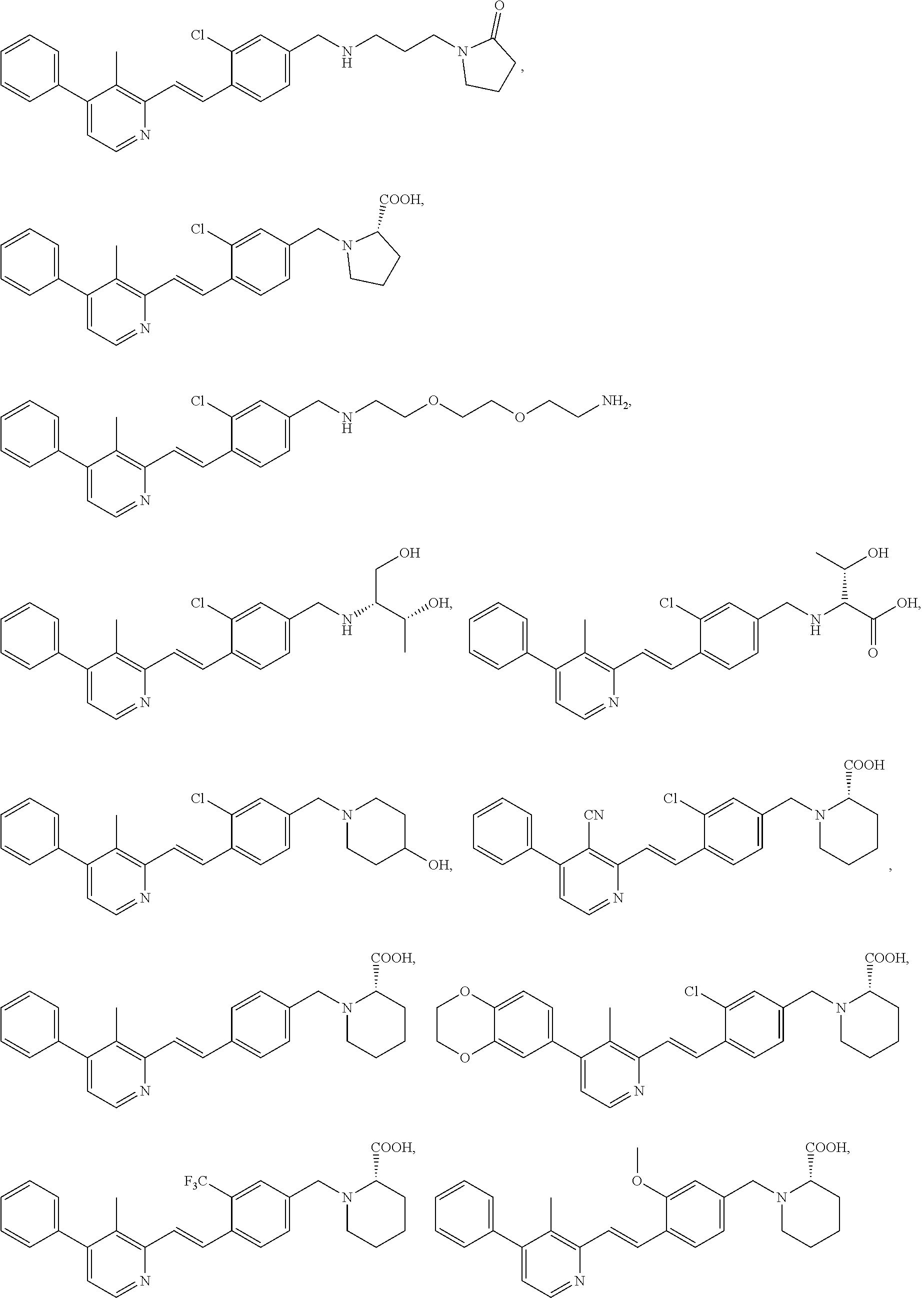

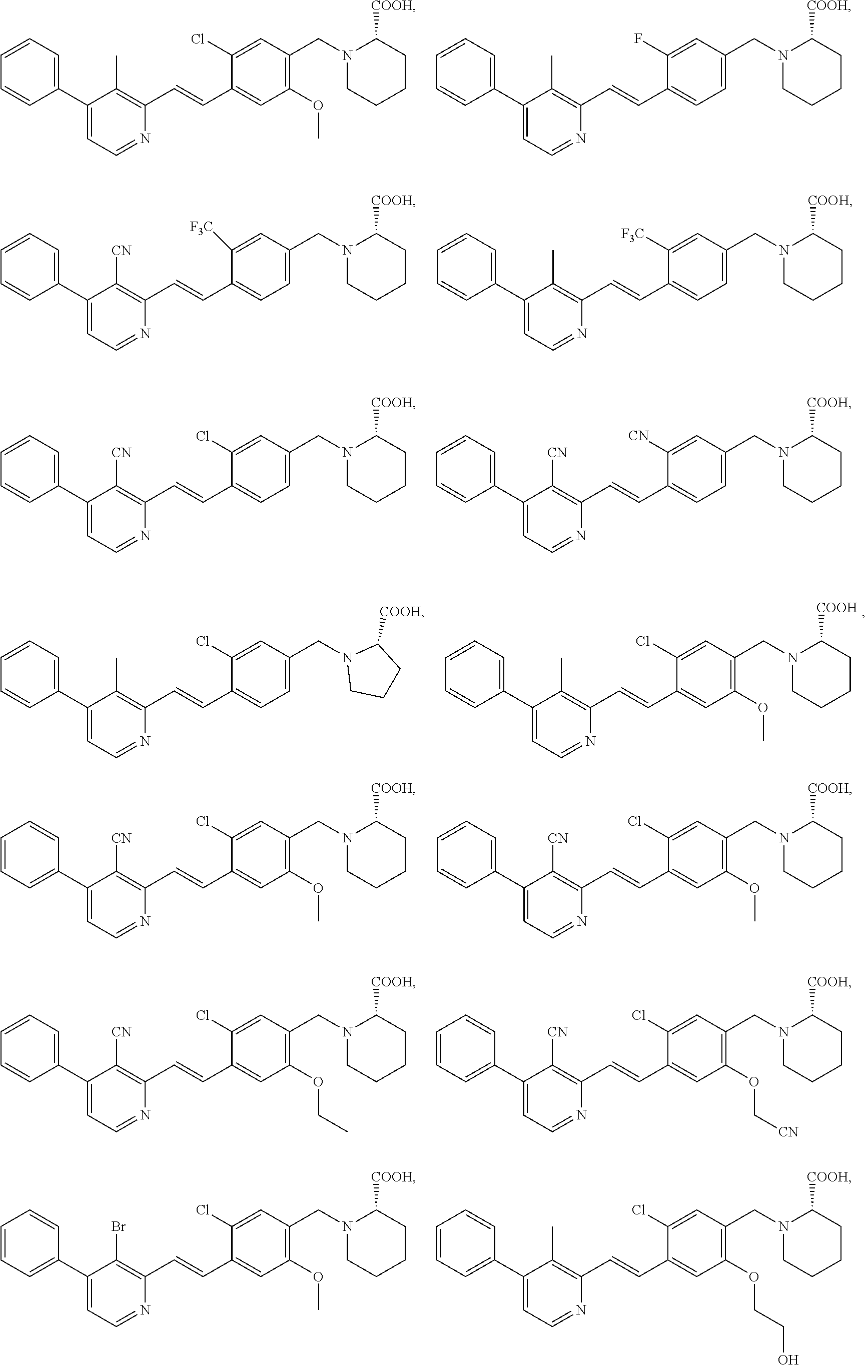

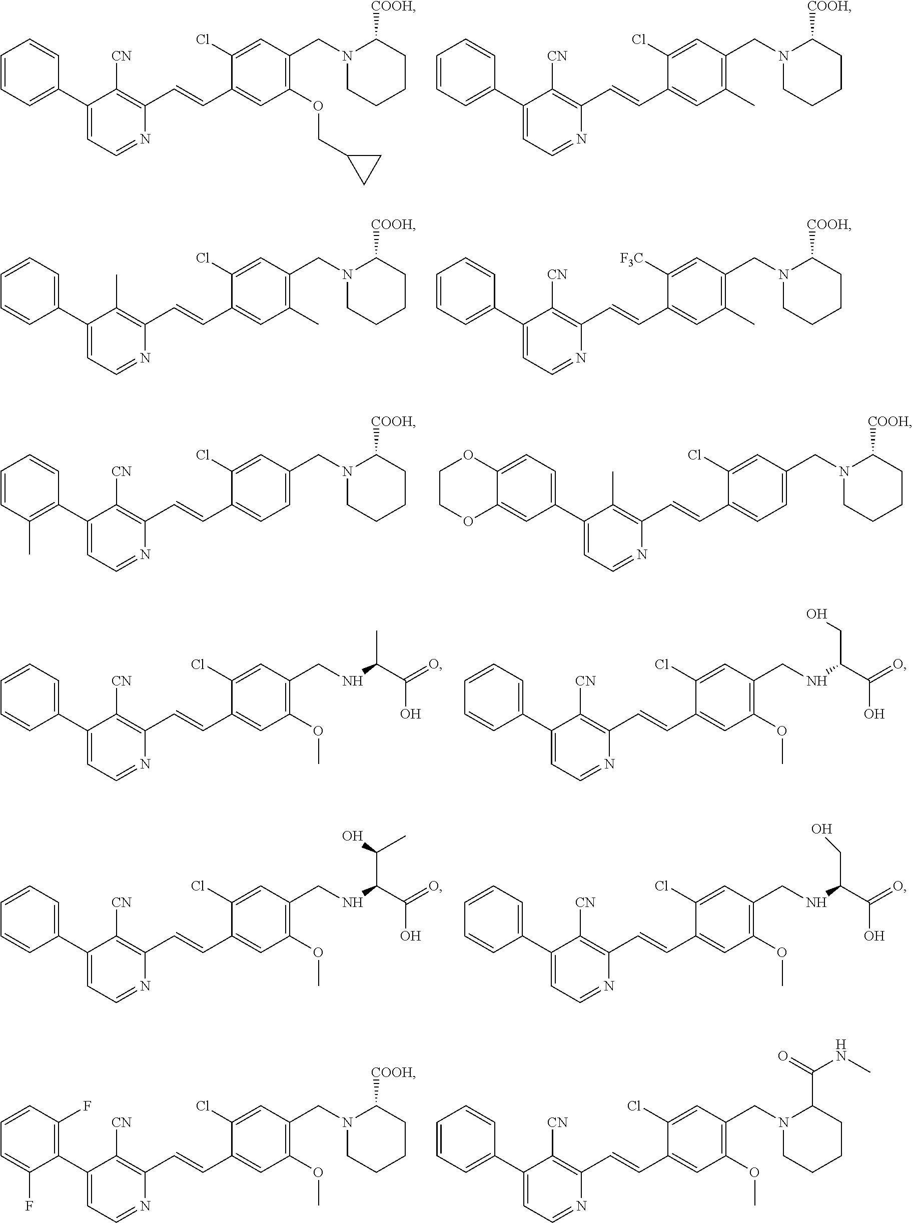

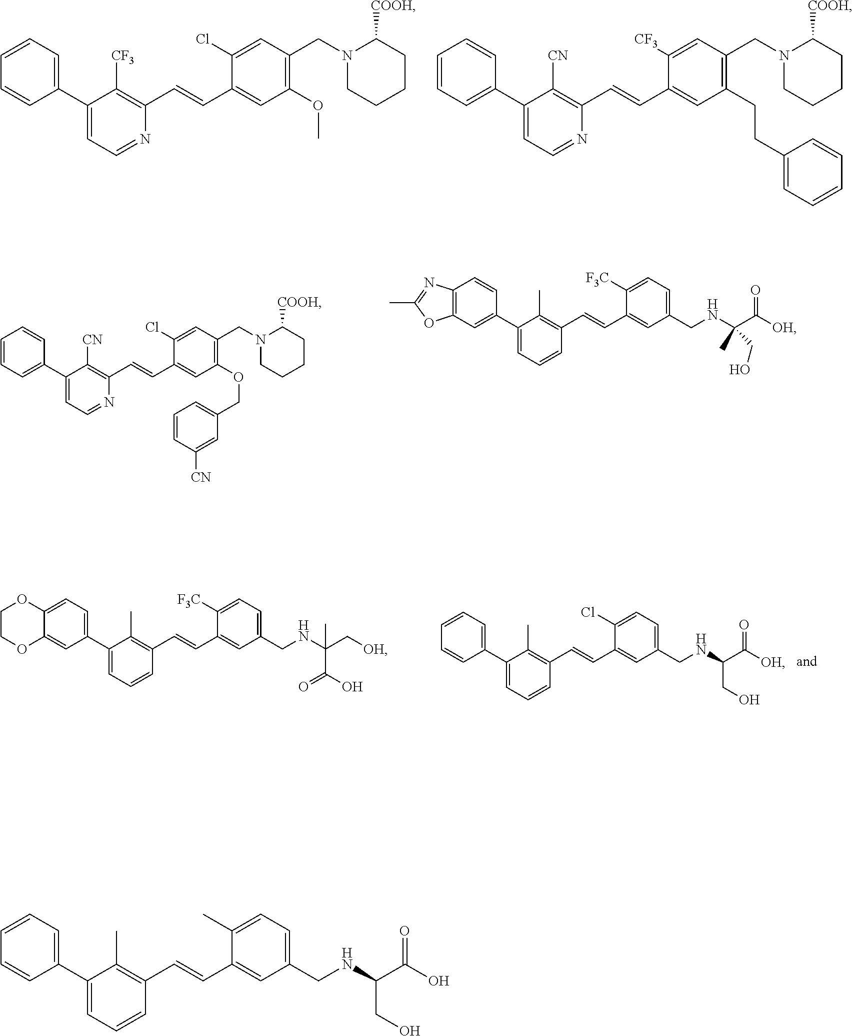

in free or pharmaceutically acceptable salt form.

1.11 Any foregoing method, wherein the small molecule inhibitor is

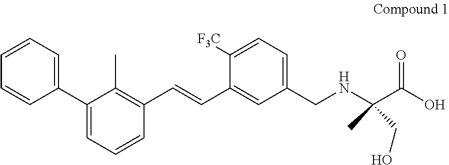 [SMILES: Cc1c(/C=C/c2cc(CN[C@@](C)(CO)C(=O)O)ccc2C(F)(F)F)cccc1-c1ccccc1]

in free or pharmaceutically acceptable salt form.

1.12 Any foregoing method wherein the small molecule inhibitor in free or pharmaceutically acceptable salt form is in prodrug form, for example in the form of a physiologically hydrolysable and acceptable ester (e.g., wherein by the term “physiologically hydrolysable and acceptable ester” refers to esters of such compounds comprising hydroxy or carboxy groups which are hydrolysable under physiological conditions to yield acids or alcohols respectively, which are themselves physiologically tolerable at doses to be administered, for example, amino acid esters); e.g., a compound as disclosed in any of Methods 1.8-1.11 wherein the small molecule inhibitor is a carboxylic acid or alcohol and the prodrug is a physiologically hydrolysable and acceptable ester of such small molecule inhibitor carboxylic acid or alcohol.

1.13 Any foregoing method, wherein the cancer is bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, unresectable or metastatic melanoma, metastatic non-small cell lung cancer, advanced renal cell carcinoma, Relapsed or Progressed Classical Hodgkin Lymphoma, Recurrent or Metastatic Squamous Cell Carcinoma of the Head and Neck, Locally Advanced or Metastatic Urothelial Carcinoma, Advanced Hepatocellular Carcinoma, Metastatic Small Cell Lung Cancer, MSI-H/dMMR Metastatic Colorectal Cancer, Primary Mediastinal Large B-Cell Lymphoma, gastric or gastroesophageal junction adenocarcinoma, cervical cancer, Hepatocellular Carcinoma, or Merkel Cell Carcinoma, cancer having interstitial fluid pressure (IFP) of at least 10 mmHg or combinations of the cancers.

1.14 Any foregoing method, wherein the cancer is unresectable or metastatic melanoma, metastatic non-small cell lung cancer, advanced renal cell carcinoma, Relapsed or Progressed Classical Hodgkin Lymphoma, Recurrent or Metastatic Squamous Cell Carcinoma of the Head and Neck, Locally Advanced or Metastatic Urothelial Carcinoma, Advanced Hepatocellular Carcinoma, Metastatic Small Cell Lung Cancer, MSI-H/dMMR Metastatic Colorectal Cancer, Primary Mediastinal Large B-Cell Lymphoma, gastric or gastroesophageal junction adenocarcinoma, cervical cancer, Hepatocellular Carcinoma, or Merkel Cell Carcinoma.

1.15 Any foregoing method, wherein the cancer is cervical carcinomas, renal cell carcinoma, melanomas, breast cancer, colorectal cancer, or head and neck squamous cell carcinoma (HNSCC).

1.16 Any foregoing method, wherein the cancer is breast cancer.

1.17 Any foregoing method, wherein the cancer is melanomas.

1.18 Any foregoing method, wherein the cancer is colorectal cancer.

1.19 Any foregoing method, wherein the cancer has interstitial fluid pressure (IFP) of at least 10 mmHg.

1.20 Any foregoing method, wherein the subject is a human.

1.21 Any foregoing method, wherein the small molecule inhibitor is administered orally.

1.22 Any foregoing method, wherein the small molecule inhibitor is administered at a total dose of 20-300 mg/kg, 30-240 mg/kg, 40-200 mg/kg, 50-190 mg/kg, 60-180 mg/kg, 70-170 mg/kg, 80-160 mg/kg, 90-150 mg/kg or 100-140 mg/kg per day.

1.23 Any of Methods 1-1.22, wherein the small molecule inhibitor is administered at an amount of about 10-150 mg/kg, 15-120 mg/kg, 20-100 mg/kg, 30-90 mg/kg, or 40-80 mg/kg body weight twice a day (BID).

1.24 Any of Methods 1-1.22, wherein the small molecule inhibitor is administered at an amount of about 30 mg/kg, 35 mg/kg, 40 mg/kg, 45 mg/kg, 50 mg/kg, 55 mg/kg, 60 mg/kg, 65 mg/kg, 70 mg/kg, 75, mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg or 150 mg/kg body weight BID.

1.25 Any of Methods 1-1.22, wherein the small molecule inhibitor is administered at an amount of about 30 mg/kg body weight BID.

1.26 Any of Methods 1-1.22, wherein the small molecule inhibitor is administered at an amount of about 60 mg/kg body weight BID.

1.27 Any of Methods 1-1.22, wherein the small molecule inhibitor is administered at an amount of about 90 mg/kg body weight BID.

1.28 Any of Methods 1-1.22, wherein the small molecule inhibitor is administered at an amount of about 120 mg/kg body weight BID.

1.29 Any foregoing method, wherein the small molecule inhibitor is simultaneously administered with the antibody.

1.30 Any foregoing method, wherein the small molecule inhibitor and the antibody are administered sequentially.

1.31 Any foregoing method, wherein the antibody is a monoclonal antibody.

1.32 Any foregoing method, wherein the antibody is pembrolizumab, nivolumab, cemiplimab, toripalimab, camrelizumab or sintilimab.

1.33 Any foregoing method, wherein the antibody is pembrolizumab.

1.34 Any foregoing method, wherein the antibody is nivolumab.

1.35 Any foregoing method, wherein the antibody is administered intravenously or subcutaneously.

1.36 Any foregoing method, wherein the antibody is administered intravenously.

1.37 Any foregoing method, wherein the antibody is administered at an amount of 0.1-50 mg/kg, 0.2-10 mg/kg, 0.3-5 mg/kg, 0.4-5 mg/kg, 0.5-5 mg/kg, 0.6-4 mg/kg, 0.6-3 mg/kg, 0.6-2 mg/kg, 0.8-4 mg/kg, 0.8-3 mg/kg, 0.8-2 mg/kg, 1-10 mg/kg, 1-5 mg/kg, 1-4 mg/kg, 1-3 mg/kg, 1-2 mg/kg or 2-3 mg/kg twice a week (BIW), once every week, once every two weeks, once every three weeks or once every four weeks.

1.38 Any foregoing method wherein the dosage of the antibody used is lower than the dosage of the antibody when used as monotherapy.

1.39 Any foregoing method wherein the antibody is nivolumab and wherein the nivolumab is administered as an intravenous infusion over 30 minutes at a dosage of up to 240 mg every 2 weeks, or up to 480 mg every 4 weeks.

1.40 Any foregoing method wherein the antibody is pembrolizumab and wherein the pembrolizumab is administered as an intravenous infusion over 30 minutes, at a dosage of up to 200 mg, every 3 weeks, until disease progression or unacceptable toxicity.

1.41 Any foregoing method, wherein the subject has previously received cancer treatment.

1.42 Method 1.41, wherein the previous cancer treatment is chemotherapy.

1.43 Method 1.42, wherein the chemotherapy comprises a platinum containing chemotherapeutic agent.

1.44 Method 1.42, wherein the chemotherapy is platinum containing doublet chemotherapy.

1.45 Method 1.41, wherein the previous cancer treatment comprises administering the anti-PD-1 antibody without co-administering the small molecule inhibitor of the PD-1/PD-L1 interaction.

1.46 Any of Methods 1.41-1.45, wherein the subject is not responsive to the previous cancer treatment.

1.47 Method 1.46, wherein the previous cancer treatment leads to a side effect and wherein a reduced dose of the anti-PD-1 antibody than the previous cancer treatment is co-administered with the small molecule inhibitor of the PD-1/PD-L1 interaction.

1.48 Any foregoing method, wherein the subject does not have a history of significant autoimmune disease.

1.49 Any foregoing method, wherein the subject has not received organ or bone marrow transplants.

1.50 Any foregoing method further comprising administration of an addition anti-cancer agent (e.g., anti CTLA-4 antibody (e.g., ipilimumab), kinase inhibitors (e.g., an inhibitor binding to vascular endothelial growth factor (VEGF)), or chemotherapeutic agents (e.g., Ziv-aflibercept, Brentuximab Vedotin, Deferiprone, Gemcitabine, Pralatrexate, Ganciclovir, Valganciclovir, Thalidomide, Romidepsin, Boceprevir, Decitabine, Imatinib, Topotecan, Lenalidomide, Paclitaxel, Olanzapine, Irinotecan, Paliperidone, Interferons, Lipopolysaccharide, tamoxifen, Flecainide (a class 1C cardiac antiarrhythmic drug), Phenytoin, Indomethacin, Propylthiouracil, Carbimazole, Chlorpromazine, Trimethoprim/sulfamethoxazole (cotrimoxazole), Clozapine, Ticlodipine, and their derivatives, Cyclophosphamide, Mechlorethanime, Chlorambucil, Melphalan, Carmustine (BCNU), Lomustine (CCNU), Procarbazine, Dacarbazine (DTIC), Altretamine, Cisplatin, Carboplatin, Actinomycin D, Etoposide, Doxorubicin & daunorubicin, 6-Mercaptopurine, 6-Thioguanine, Idarubicin, Epirubicin, Mitoxantrone, Azathioprine, 2-Chloro deoxyadenosine, Hydroxyurea, Methotrexate, 5-Fluorouracil, Cytosine arabinoside, Azacytidine, Fludarabine phosphate, Vincristine, Vinblastine, Vinorelbine, Docetaxel, Pemetrexed, Nab-paclitaxel, Dasatinib, Paralatrexate, Sunitinib, or Oxaliplatin)).

In another aspect, the invention provides a use of a small molecule inhibitor of the PD-1/PD-L1 interaction as disclosed herein (e.g., as disclosed in any of Methods 1.8-1.11 supra) in the manufacture of a medicament for use in combination with an anti-PD-1 antibody (e.g., pembrolizumab, nivolumab, cemiplimab, toripalimab, camrelizumab or sintilimab, e.g., pembrolizumab or nivolumab) in treating a cancer, wherein the small molecule inhibitor is not a protein, e.g., in accordance with any of Methods 1, et seq.

In another aspect, the invention provides the use of an anti-PD-1 antibody (e.g., pembrolizumab, nivolumab, cemiplimab, toripalimab, camrelizumab or sintilimab, e.g., pembrolizumab or nivolumab) in the manufacture of a medicament for use in combination with a small molecule inhibitor of the PD-1/PD-L1 interaction as disclosed herein (e.g., as disclosed in any of Methods 1.8-1.11 supra), wherein the small molecule inhibitor is not a protein, e.g., in accordance with any of Methods, 1 et seq.

In another aspect, the invention provides the use of an anti-PD-1 antibody (e.g., pembrolizumab, nivolumab, cemiplimab, toripalimab, camrelizumab or sintilimab, e.g., pembrolizumab or nivolumab) in the manufacture of a medicament for use in combination with a small molecule inhibitor of the PD-1/PD-L1 interaction as disclosed herein (e.g., as disclosed in any of Methods 1.8-1.11 supra) in treating a cancer, wherein the small molecule inhibitor is not a protein, e.g., in accordance with any of Methods, 1 et seq.

In another aspect, the invention provides a small molecule inhibitor of the PD-1/PD-L1 interaction as disclosed herein (e.g., as disclosed in any of Methods 1.8-1.11 supra) for use in combination with an anti-PD-1 antibody (e.g., pembrolizumab, nivolumab, cemiplimab, toripalimab, camrelizumab or sintilimab, e.g., pembrolizumab or nivolumab) in treating a cancer, wherein the small molecule inhibitor is not a protein, e.g., in accordance with any of Methods 1 et seq.

In another aspect, the invention provides an anti-PD-1 antibody (e.g., pembrolizumab, nivolumab, cemiplimab, toripalimab, camrelizumab or sintilimab, e.g., pembrolizumab or nivolumab) for use in combination with a small molecule inhibitor of the PD-1/PD-L1 interaction as disclosed herein (e.g., as disclosed in any of Methods 1.8-1.11 supra), wherein the small molecule inhibitor is not a protein, e.g., in accordance with any of Methods 1 et seq.

In another aspect, the invention provides an anti-PD-1 antibody (e.g., pembrolizumab, nivolumab, cemiplimab, toripalimab, camrelizumab or sintilimab, e.g., pembrolizumab or nivolumab) for use in combination with a small molecule inhibitor of the PD-1/PD-L1 interaction as disclosed herein (e.g., as disclosed in any of Methods 1.8-1.11 supra) in treating a cancer, wherein the small molecule inhibitor is not a protein, e.g., in accordance with any of Methods 1 et seq.

The small molecule inhibitors of the PD-1/PD-L1 interaction as disclosed herein can be synthesized by methods known in the art, e.g., methods disclosed in WO2018006795 and WO2019128918.

In some embodiments, the invention provides pharmaceutical compositions comprising a therapeutically effective amount of a small molecule inhibitor of the PD-1/PD-L1 interaction of the present invention disclosed hereinabove (e.g., as disclosed in any of Methods 1.8-1.11 supra) for use in combination with a therapeutically effective amount of an anti-PD-1 antibody (e.g., pembrolizumab, nivolumab, cemiplimab, toripalimab, camrelizumab or sintilimab, e.g., pembrolizumab or nivolumab) in treating a cancer.

The pharmaceutical compositions for use in combination with an anti-PD-1 antibody may be used in treating various cancers, e.g., bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, unresectable or metastatic melanoma, metastatic non-small cell lung cancer, advanced renal cell carcinoma, Relapsed or Progressed Classical Hodgkin Lymphoma, Recurrent or Metastatic Squamous Cell Carcinoma of the Head and Neck, Locally Advanced or Metastatic Urothelial Carcinoma, Advanced Hepatocellular Carcinoma, Metastatic Small Cell Lung Cancer, MSI-H/dMMR Metastatic Colorectal Cancer, Primary Mediastinal Large B-Cell Lymphoma, gastric or gastroesophageal junction adenocarcinoma, Merkel Cell Carcinoma, cancer having interstitial fluid pressure (IFP) of at least 10 mmHg, cervical cancer or combinations of the cancers.

In some embodiments, the combination of a small molecule inhibitor of the PD-1/PD-L1 interaction and an anti-PD-1 antibody may be administered to a subject that has previously received cancer treatment, e.g., chemotherapy or anti-PD-1 antibody therapy without co-administration of the small molecule inhibitor of the PD-1/PD-L1 interaction. In some embodiments, the combination therapy of the present invention is used to a patient that is not responsive to the previous cancer treatment. For a subject in which the previous anti-PD-1 antibody therapy has led to a side effect, a reduced dose of the anti-PD-1 antibody than the previous anti-PD-1 antibody therapy may be co-administered with the small molecule inhibitor of the PD-1/PD-L1 interaction.

The pharmaceutical composition comprising a small molecule inhibitor of the PD-1/PD-L1 interaction may include conventional pharmaceutically acceptable carriers, excipients, or diluents. The “therapeutically effective amount” can be determined according to the subject's category, age, sex, severity and type of disease, activity of drug, sensitivity to drug, administration time, administration route, excretion rate, and so forth. The amount of the small molecule inhibitor in the pharmaceutical composition can be widely varied without specific limitation, and may be specifically 0.00001 weight % to 10 weight %, e.g., 0.0001 weight % to 5 weight %, or 0.001 weight % to 1 weight % with respect to the total amount of the composition. The pharmaceutical composition may be formulated into solid, liquid, gel or suspension form for oral or non-oral administration, for example, tablet, bolus, powder, granule, capsule such as hard or soft gelatin capsule, emulsion, suspension, syrup, emulsifiable concentrate, sterilized aqueous solution, non-aqueous solution, freeze-dried formulation, suppository, and so on. For oral administration, the pharmaceutical composition comprising the small molecule inhibitor of the PD-1/PD-L1 interaction may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 0.1 to 1000 mg.

Any pharmaceutical composition contemplated herein can, for example, be delivered orally via any acceptable and suitable oral preparations. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the disclosure can contain at least one agent selected from sweetening agents, flavoring agents, coloring agents, demulcents, antioxidants, and preserving agents.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

A sterile injectable oil-in-water microemulsion can, for example, be prepared by 1) dissolving a small molecule inhibitor of the PD-1/PD-L1 interaction in an oily phase, such as, for example, a mixture of soybean oil and lecithin; 2) combining the small molecule inhibitor containing oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers, adjuvants, and vehicles that may be used in the pharmaceutical compositions of this disclosure include, but are not limited to, ion exchangers, alumina, aluminum stearate, lecithin, self-emulsifying drug delivery systems (SEDDS) such as d-alpha-tocopherol polyethyleneglycol 1000 succinate, surfactants used in pharmaceutical dosage forms such as Tweens, polyethoxylated castor oil such as CREMOPHOR surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active small molecule inhibitor of the PD-1/PD-L1 interaction of this disclosure can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

The amounts of small molecule inhibitors that are administered and the dosage regimen for treating a disease condition with the small molecule inhibitors and/or compositions of this disclosure depends on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods. A daily dose of about 0.001 to 250 mg/kg body weight, preferably between about 0.0025 and about 150 mg/kg body weight and most preferably between about 0.005 to 120 mg/kg body weight, may be appropriate. The daily dose can be administered in one to four doses per day. Other dosing schedules include one dose per week and one dose per two-day cycle. In some embodiments, the small molecule inhibitor may be administered at a total dose of 20-300 mg/kg, 30-240 mg/kg, 40-200 mg/kg, 50-190 mg/kg, 60-180 mg/kg, 70-170 mg/kg, 80-160 mg/kg, 90-150 mg/kg or 100-140 mg/kg per day. In other embodiments, the small molecule inhibitor may be administered at an amount of about 10-150 mg/kg, 15-120 mg/kg, 20-100 mg/kg, 30-90 mg/kg, or 40-80 mg/kg body weight twice a day (BID).

For therapeutic purposes, the small molecule inhibitors of the PD-1/PD-L1 interaction are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

Anti-PD-1 antibodies may be formulated together with a pharmaceutically acceptable carrier, which includes any solvents, dispersion media, isotonic and absorption delaying agents, and the like that are physiologically compatible. The carrier may be suitable for intravenous, intramuscular, subcutaneous, parenteral, rectal, spinal or epidermal administration (e.g., by injection or infusion). The composition comprising anti-PD-1 antibodies can be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and the therapeutic application. Typical preferred compositions are in the form of injectable or infusible solutions. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

Dosage regimens of anti-PD-1 antibodies may be adjusted to provide the desired optimal response (e.g., a therapeutic response). For example, a single bolus may be administered, several divided doses may be administered over time, or the dose may be reduced or increased proportionally as indicated by the demands of the therapeutic situation. When anti-PD-1 antibodies are co-administered with small molecule inhibitors of the PD-1/PD-L1 interaction, the dose of anti-PD-1 antibodies may be reduced. In some embodiment, anti-PD-1 antibodies may be administered at a dose of 0.1-50 mg/kg, 0.2-10 mg/kg, 0.3-5 mg/kg, 0.4-5 mg/kg, 0.5-5 mg/kg, 0.6-4 mg/kg, 0.6-3 mg/kg, 0.6-2 mg/kg, 0.8-4 mg/kg, 0.8-3 mg/kg, 0.8-2 mg/kg, 1-10 mg/kg, 1-5 mg/kg, 1-4 mg/kg, 1-3 mg/kg, 1-2 mg/kg or 2-3 mg/kg twice a week (BIW), once every week, once every two weeks, once every three weeks or once every four weeks.

Small molecule inhibitors of the PD-1/PD-L1 interaction and anti-PD-1 antibodies may be administered in combination with standard cancer treatments, e.g., one or more other therapeutic agents, e.g., a cytotoxic agent, a radiotoxic agent or an immunosuppressive agent. The combination of a small molecule inhibitor of the PD-1/PD-L1 interaction and an anti-PD-1 antibody may be administered before, after or concurrently with the other therapeutic agent or can be co-administered with other known therapies, e.g., an anti-cancer therapy, e.g., radiation. Such therapeutic agents include, among others, anti-neoplastic agents such as doxorubicin (adriamycin), cisplatin bleomycin sulfate, carmustine, chlorambucil, decarbazine and cyclophosphamide hydroxyurea which, by themselves, are only effective at levels which are toxic or subtoxic to a patient. Cisplatin is intravenously administered as a 100 mg/dose once every four weeks and adriamycin is intravenously administered as a 60-75 mg/ml dose once every 21 days. Co-administration of different chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibodies.

PD-L1 blockade may be effectively combined with chemotherapeutic regimes. In these instances, it may be possible to reduce the dose of chemotherapeutic reagent administered. Chemotherapeutic agents that may be used in combination of a small molecule inhibitor of the PD-1/PD-L1 interaction and an anti-PD-1 antibody include but are not limited to Ziv-aflibercept, Brentuximab Vedotin, Deferiprone, Gemcitabine, Pralatrexate, Ganciclovir, Valganciclovir, Thalidomide, Romidepsin, Boceprevir, Decitabine, Imatinib, Topotecan, Lenalidomide, Paclitaxel, Olanzapine, Irinotecan, Paliperidone, Interferons, Lipopolysaccharide, tamoxifen, Flecainide (a class 1C cardiac antiarrhythmic drug), Phenytoin, Indomethacin, Propylthiouracil, Carbimazole, Chlorpromazine, Trimethoprim/sulfamethoxazole (cotrimoxazole), Clozapine, Ticlodipine, and their derivatives, Cyclophosphamide, Mechlorethanime, Chlorambucil, Melphalan, Carmustine (BCNU), Lomustine (CCNU), Procarbazine, Dacarbazine (DTIC), Altretamine, Cisplatin, Carboplatin, Actinomycin D, Etoposide, Topotecan, Irinotecan, Doxorubicin & daunorubicin, 6-Mercaptopurine, 6-Thioguanine, Idarubicin, Epirubicin, Mitoxantrone, Azathioprine, 2-Chloro deoxyadenosine, Hydroxyurea, Methotrexate, 5-Fluorouracil, Cytosine arabinoside, Azacytidine, Gemcitabine, Fludarabine phosphate, Vincristine, Vinblastine, Vinorelbine, Paclitaxel, Docetaxel, Tamoxifen, Pemetrexed, Nab-paclitaxel, Dasatinib, Paralatrexate, Decitabine, Romidepsin, Imatinib, Lenalidomide, Sunitinib, Oxaliplatin, and Thalidomide.

Tumors evade host immune surveillance by a large variety of mechanisms. Many of these mechanisms may be overcome by the inactivation of proteins which are expressed by the tumors and which are immunosuppressive. These include among others TGF-beta (Kehrl, J. et al. (1986) J. Exp. Med. 163: 1037-1050), IL-10 (Howard, M. & O'Garra, A. (1992) Immunology Today 13: 198-200), and Fas ligand (Hahne, M. et al. (1996) Science 274: 1363-1365). Macrocyclic peptides to each of these entities may be used in combination with the compounds of this disclosure to counteract the effects of the immunosuppressive agent and favor tumor immune responses by the host.

Macrocyclic peptides that activate host immune responsiveness can be used in combination with small molecule inhibitors of the PD-1/PD-L1 interaction and anti PD-1 antibodies. These include molecules on the surface of dendritic cells which activate DC function and antigen presentation. Anti-CD40 macrocyclic peptides are able to substitute effectively for T cell helper activity (Ridge, J. et al. (1998) Nature 393: 474-478) and can be used in conjunction with PD-1 macrocyclic peptides (Ito, N. et al. (2000) Immunobiology 201 (5) 527-40). Activating macrocyclic peptides to T cell costimulatory molecules such as CTLA-4 (e.g., U.S. Pat. No. 5,811,097), OX-40 (Weinberg, A. et al. (2000) Immunol 164: 2160-2169), 4-1BB (Melero, I. et al. (1997) Nature Medicine 3: 682-685 (1997), and ICOS (Hutloff, A. et al. (1999) Nature 397: 262-266) may also provide for increased levels of T cell activation.

Vascular endothelial growth factor (VEGF) is one of the most important proteins that promote angiogenesis, which is a tightly regulated process of developing new blood vessels from a pre-existing vascular network (Ferrara, N., (2004), Endocrine Reviews, 25(4): 581-611). Angiogenesis is required during development and normal physiological processes such as wound healing, and is also involved in a number of disease pathogenesis, including AMD, RA, Diabetic Retinopathy, tumor growth and metastasis. Inhibition of angiogenesis has been shown to be effective in therapeutic applications.

The above other therapeutic agents, when employed in combination with small molecule inhibitors of the PD-1/PD-L1 interaction and anti-PD-1 antibodies, may be used, for example, in those amounts indicated in the Physicians' Desk Reference (PDR) or as otherwise determined by one of ordinary skill in the art. In the methods of the present disclosure, such other therapeutic agent(s) may be administered prior to, simultaneously with, or following the administration of the small molecule inhibitor of the PD-1/PD-L1 interaction and anti-PD-1 antibody as disclosed in Method 1 et seq.

### DETAILED DESCRIPTION OF THE PREFERRED EMBODIMENT

The disclosure is further defined in the following Examples. It should be understood that the Examples are given by way of illustration only. From the above discussion and the Examples, one skilled in the art can ascertain the essential characteristics of the disclosure, and without departing from the spirit and scope thereof, can make various changes and modifications to adapt the disclosure to various uses and conditions. As a result, the disclosure is not limited by the illustrative examples set forth herein below, but rather is defined by the claims appended hereto.

### Example 1: In Vitro Binding Studies of Compound 1

Biological Assay: The ability of Compound 1 to bind to PD-L1 is investigated using a PD-1/PD-L1 Homogenous Time-Resolved Fluorescence (HTRF) binding assay.

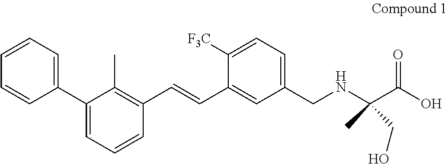 [SMILES: Cc1c(/C=C/c2cc(CN[C@@](C)(CO)C(=O)O)ccc2C(F)(F)F)cccc1-c1ccccc1]

All binding studies are performed in an HTRF assay buffer consisting of dPBS supplemented with 0.1% (w/v) bovine serum albumin and 0.05% (v/v) Tween-20. For the PD-1-Ig/PD-L1-His binding assay, inhibitors are pre-incubated with PD-L1-His (10 nM final) for 15 m in 4 μl of assay buffer, followed by addition of PD-1-Ig (20 nM final) in 1 μl of assay buffer and further incubation for 15 m. PD-L1 from either human, cynomolgus, or mouse are used. HTRF detection is achieved using europium crypate-labeled anti-Ig (1 nM final) and allophycocyanin (APC) labeled anti-His (20 nM final). Antibodies are diluted in HTRF detection buffer and 5 μl is dispensed on top of binding reaction. The reaction mixture is allowed to equilibrate for 30 minutes and signal (665 nm/620 nm ratio) is obtained using an EnVision fluorometer. Additional binding assays are established between PD-1-Ig/PD-L2-His (20 & 5 nM, respectively), CD80-His/PD-L1-Ig (100 & 10 nM, respectively) and CD80-His/CTLA4-Ig (10 & 5 nM, respectively). Competition studies between biotinylated polypeptide (AISGGGGSTYYADSVKD) and human PD-L1-His are performed as follows. Inhibitors are pre-incubated with PD-L1-His (10 nM final) for 60 m in 4 .mu·l of assay buffer followed by addition of biotinylated polypeptide (0.5 nM final) in 1 .mu·l of assay buffer. Binding is allowed to equilibrate for 30 m followed by addition of europium crypated labeled Strepatavidin (2.5 pM final) and APC-labeled anti-His (20 nM final) in 5 μl of HTRF buffer. The reaction is allowed to equilibrate for 30 m and signal (665 nm/620 nm ratio) is obtained using an EnVision fluorometer. In the HTRF assay, Compound 1 potently inhibits the binding between hPD-1 and hPD-L1 with IC₅₀ of 19 nM.

To measure the cellular activity of the compound, a protocol of Activation of T-Cell Suppressed by PD-L1 is used. In this protocol, human Hep3B cells are stably transfected with human PD-L1. The human T cells containing PD-1 are inactivated by co-culturing with these PD-L1 transfected cells. Then anti-PD-1 antibody Keytruda® (pembrolizumab) is selected as the reference to profile the compound for its activation of PD-L1 suppressed human T-cells. In a dose-dependent manner, Compound 1 effectively restores the activation of the PD-L1 suppressed human T cells, as indicated by the increase of cytokine IFN-γ. Keytruda is used as a positive control.

### Example 2: In Vivo Test of Anti-Tumor Efficacy of Compound 1 in the Subcutaneous 4T1 Murine Breast Cancer Model in BALB/c Mice

The 4T1 murine mammary carcinoma is a transplantable tumor cell line that is highly tumorigenic, invasive and able spontaneously to metastasize from the primary tumor in the mammary gland to multiple distant sites including lymph nodes, blood, liver, lung, brain, and bone.

Materials required for the experiment: Antibody: mouse PD-1 antibody, Product specifications: 7.09 mg/mL (50 mg/mL), Lot No.: 695318A1 purchased from BioXcell, storage at 4° C. Experiment animal: 60 BALB/C mice, female, 6-8 weeks old, 20-23 g, purchased from Shanghai Lingchang Biotechnology Co. Ltd. Formulation material: castor oil (Cremophor RH40), CAS No.: 61788-85-0, Lot No.: 29761847G0, purchased from Shanghai Xietai Chemical Co. Ltd.; β-cyclodextrin (SBE-β-CD), CAS No.: 128446-35-5, Lot No.: 20180110, purchased from Shanghai Shaoyuan Chemical Co. Ltd.; RPMI-1640 culture medium, Art. No.: 1869036, Lot No.: 11875-093, purchased from Gibco Co. Ltd.; PBS, Art. No.: SH30256.01, Lot No.: AB10141338, purchased from HyClone Co. Ltd.; Fetal bovine serum: CAS No.: 10099-141, Lot No.: 1966174C, purchased from Gibco Co. Ltd.

Cell preparation and implantation: 4T1 cells (CRL2539™) are cultured with RPMI 1640 supplemented with 10% heat inactivated FBS at 37° C. in 5% CO₂ incubator. Cells are passaged 3 times a week. Cells are harvested, counted and passaged, inoculated when around 70% confluent.

Tumor cell inoculation and group administration: The 50 uL cell suspension containing 1×10⁵ 4T1 tumor cells (cells suspended in base RPMI-1640 medium) is inoculated into the fourth fat pad of the left abdomen of mice. On the second day after inoculation, according to the order of tumor inoculation, stratified randomization is used to group and start the administration on the day of grouping.

Preparation of test substances: Preparation of formulation: 490 mL of sterile water is added into the volumetric flask with magnetic stirring to have a vortex. 100 g of castor oil (Cremophor RH40) is added with a spoon slowly into the vortex and the solution is kept stirring. 200 g of β-cyclodextrin (SBE-β-CD) is added while the solution is kept stirring until the solution is clear, and the total volume is set to 1000 mL, which contained 10% (w/v) Cremophor RH40+20% (w/v) an aqueous solution of SBE-β-CD.

Preparation of Compound Suspension: 178.88 mg compound is weighed and 14.817 mL 10% (w/v) Cremophor RH40+20% (w/v) SBE-β-CD aqueous solution are added. The suspension solution with a concentration of 12.0 mg/mL is obtained by fully mixing with magnetic stirring. 7.0 mL of the compound suspension solution with concentration of 12.0 mg/mL is pipetted and 7.0 mL aqueous formulation solution is added. The suspension solution with a concentration of 6.0 mg/mL is obtained by fully mixing with magnetic stirring. 7.0 mL of the compound suspension solution with concentration of 6.0 mg/mL is pipetted and 7.0 mL aqueous formulation solution is added. The suspension solution with a concentration of 3.0 mg/mL is obtained by fully mixing with magnetic stirring. 7.0 mL of the compound suspension solution with concentration of 3.0 mg/mL is pipetted and 7.0 mL aqueous formulation solution is added. The suspension solution with a concentration of 1.5 mg/mL is obtained by fully mixing with magnetic stirring. The compound suspension solution is prepared once a day.

Preparation of mPD-1 Antibody: 0.339 mL mPD-1 antibody (7.09 mg/mL) original solution is pipetted and 2.061 mL PBS solution is added. The solution is fully mixed and the final concentration of 1 mg/mL solution is obtained.

Procedure: The mice in the vehicle group are weighed and recorded in the electronic balance according to their numbers. The mice in the vehicle group are given prepared formulation solution twice a day by oral administration according to their body weight with a capacity of 0.1 mL/10 g.

The mice in the antibody (10 mg/kg) group are weighed and recorded in the electronic balance according to their numbers. The mice in the antibody group are given prepared antibody solution twice a week by IP administration according to their body weight with a capacity of 0.1 ml/10 g.

The mice in the compound (15 mg/kg) group are weighed and recorded in the electronic balance according to their numbers. The mice in this group are given prepared compound suspension twice a day by oral administration according to their body weight with a capacity of 0.1 ml/10 g.

The mice in the compound (30 mg/kg) group are weighed and recorded in the electronic balance according to their numbers. The mice in this group are given prepared compound suspension twice a day by oral administration according to their body weight with a capacity of 0.1 ml/10 g.

The mice in the compound (60 mg/kg) group are weighed and recorded in the electronic balance according to their numbers. The mice in this group are given prepared compound suspension twice a day by oral administration according to their body weight with a capacity of 0.1m/10 g.

The mice in the compound (120 mg/kg) group are weighed and recorded in the electronic balance according to their numbers. The mice in this group are given prepared compound suspension twice a day by oral administration according to their body weight with a capacity of 0.1 ml/10 g.

Tumors are measured with digital vernier calipers three times a week and calculating the volume of tumors. Euthanasia is imposed if the size of the tumor exceeds 2000 mm³, or when the animal has serious disease, pain, or is unable to freely eat and drink water. The body weight of the animals is measured by electronic balance every day. Euthanasia is required when the animal is obviously thin and its weight is reduced by more than 20%. The experiment ends 20 days after compound is administered.

The tumor inhibition rate is calculated as:

TGI (%)=(1−(the volume of the tumor on the day of administration−the volume of the tumor on the first day of administration(treatment group))/(the volume of the tumor on the day of administration−the volume of the tumor on the first day (vehicle group)))×100%.

With GraphPad Prism 5.0 software, the tumor volume changes in mice are analyzed by Two-way ANOVA and compared with the vehicle group according to the Bonferroni posttests method, P<0.05 is considered to be significantly different.

In the assay, Compound 1 and mPD-1 antibody demonstrated similar efficacy in tumor growth inhibition (TGI). Furthermore, the minimum effective dose of Compound 1 is 30 mpk (p<0.05). The results are summarized in Table 1.

TABLE 1
Results of In vivo test of anti-tumor efficacy of Compound 1 in
the subcutaneous 4T1 murine breast cancer model in BALB/c mice.

| Groups | Tumor Volume (mm³) | TGI (%) | p-Value |
|---|---|---|---|
| Vehicle Control | 860.89 ± 42.52 | — | — |
| mPD-1 antibody, | 700.96 ± 39.56 | 18.58 | <0.001 |
| 10 mg/kg, IP, BIW | | | |
| Compound 1, | 689.03 ± 43.97 | 19.96 | <0.001 |
| 15 mg/kg, PO, BID | | | |
| Compound 1, | 573.9 ± 43.18 | 33.34 | <0.001 |
| 30 mg/kg, PO, BID | | | |
| Compound 1, | 548.24 ± 31.39 | 36.32 | <0.001 |
| 60 mg/kg, PO, BID | | | |
| Compound 1, | 503.16 ± 32.93 | 41.55 | <0.001 |
| 120 mg/kg, PO, BID | | | |

### Example 3 In Vivo Test of Anti-Tumor Efficacy of Compound Land mPD-1 Antibody in the B16F10 Models

Materials required for the experiment: Antibody: mouse PD-1 antibody, Product specifications: 7.09 mg/mL (50 mg/mL), Lot No.: 695318A1 purchased from BioXcell, storage at 4° C. Experimental animals 60 C57BL/6 mice, female, 6-8 weeks old, 17-21 g, purchased from Shanghai Lingchang Biotechnology Co. Ltd. Formulation materials: castor oil (Cremophor RH40), CAS No.: 61788-85-0, Lot No.: 29761847G0, purchased from Shanghai Xietai Chemical Co. Ltd.; β-cyclodextrin (SBE-β-CD), CAS No.: 128446-35-5, Lot No.: 20180110, purchased from Shanghai Shaoyuan Chemical Co. Ltd.; DMEM culture medium, Art. No.: 11995-065, Lot No.: 2025378, purchased from Gibco Co. Ltd.; PBS, Art. No.: SH30256.01, Lot No.: AB10141338, purchased from HyClone Co. Ltd.; Fetal bovine serum: Art. No.: 04-002-1A, Lot No.: 1625436, purchased from Boehringer Ingelheim Co. Ltd.; Methyl cellulose (MC), Art. No.: M7027-250G, Lot No.: 079K0054V, purchased from Sigma.

Cell preparation and implantation: The B16-F10 tumor cells (ATCC CRL-6475™) are maintained in vitro as a monolayer culture in DMEM medium supplemented with 10% heat inactivated fetal bovine serum at 37° C. in an atmosphere of 5% CO₂ in air. The tumor cells are routinely subcultured three times weekly by trypsin-EDTA treatment. The cells growing to a confluency around 70%-80% are harvested and counted for tumor inoculation.

Tumor cell inoculation and group administration: The 100 uL cell suspension containing 1×10⁶ B16F10 tumor cells (cells suspended in base DMEM medium) is inoculated into the right subcutaneous of mice. On the second day after inoculation, according to the order of tumor inoculation, stratified randomization is used to group and start the administration on the day of grouping.

Preparation of test substances: Preparation of formulation: 700 mL of sterile water is added into the volumetric flask with magnetic stirring to have a vortex. 100 g of castor oil (Cremophor RH40) is added with a spoon slowly into the vortex and the solution is kept stirring. 200 g of β-cyclodextrin (SBE-β-CD) is added while the solution is kept stirring until the solution is clear, and the total volume is set to 1000 mL, which contained 10% (w/v) Cremophor RH40+20% (w/v) an aqueous solution of SBE-β-CD.

Preparation of compound suspension: 169.16 mg compound is weighed, 14.012 mL 10% (w/v) Cremophor RH40+20% (w/v) SBE-β-CD aqueous solution are added, and the suspension solution with a concentration of 12.0 mg/mL is obtained by fully mixing with magnetic stirring. 6.0 mL of the compound suspension solution with concentration of 12.0 mg/mL is pipetted and 6.0 mL aqueous formulation solution is added. The suspension solution with a concentration of 6.0 mg/mL is obtained by fully mixing with magnetic stirring. 6.0 mL of the compound suspension solution with concentration of 6.0 mg/mL is pipetted and 6.0 mL aqueous formulation solution is added. The suspension solution with a concentration of 3.0 mg/mL is obtained by fully mixing with magnetic stirring. The compound suspension solution is prepared once a day.

Preparation of mPD-1 antibody: 0.564 mL mPD-1 antibody (7.09 mg/mL) original solution is pipetted and 3.307 mL PBS solution is added. The solution is fully mixed and the final concentration of 1 mg/mL solution is obtained.

Procedure: The mice in the vehicle group are weighed and recorded in the electronic balance according to their numbers. The mice in the vehicle group are given prepared formulation solution twice a day by oral administration according to their body weight with a capacity of 0.1 mL/10 g.

The mice in the antibody (10 mg/kg) group are weighed and recorded in the electronic balance according to their numbers. The mice in the antibody group are given prepared antibody solution twice a week by IP administration according to their body weight with a capacity of 0.1 ml/10 g.

The mice in the compound (30 mg/kg) group are weighed and recorded in the electronic balance according to their numbers. The mice in this group are given prepared compound suspension twice a day by oral administration according to their body weight with a capacity of 0.1 ml/10 g.

The mice in the compound (60 mg/kg) group are weighed and recorded in the electronic balance according to their numbers. The mice in this group are given prepared compound suspension twice a day by oral administration according to their body weight with a capacity of 0.1 ml/10 g.

The mice in the compound (120 mg/kg) group are weighed and recorded in the electronic balance according to their numbers. The mice in this group are given prepared compound suspension twice a day by oral administration according to their body weight with a capacity of 0.1 ml/10 g.

The mice in the combo group (compound, 60 mg/kg; mPD-1, 10 mg/kg) are weighed and recorded in the electronic balance according to their numbers. The mice in this group are given prepared compound suspension twice a day by oral administration according to their body weight with a capacity of 0.1 ml/10 g and mouse antibody solution twice a week by IP administration according to their body weight with a capacity of 0.1 ml/10 g.

Tumors are measured with digital vernier calipers three times a week and calculating the volume of tumors. Euthanasia is imposed if the size of the tumor exceeds 2000 mm³, or the animal has serious disease, pain, or is unable to freely eat and drink water. The body weight of the animals is measured by electronic balance every day. Euthanasia is required when the animal is obviously thin and its weight is reduced by more than 20%. The experiment ends 20 days after compound is administered.

The tumor inhibition rate is calculated as:

TGI (%)=(1−(the volume of the tumor on the day of administration−the volume of the tumor on the first day of administration (treatment group))/(the volume of the tumor on the day of administration−the volume of the tumor on the first day (vehicle group)))×100%.

Using GraphPad Prism 5.0 software, the tumor volume changes in mice are analyzed by Two-way ANOVA and compared with the vehicle group according to the Bonferroni posttests method, P<0.05 is considered to be significantly different.

The results show that Compound 1 significantly inhibits the growth of subcutaneous transplanted melanoma cell line in mice, and it is well tolerated in C57BL/6 mice without obvious adverse reactions. Furthermore, the results show that the combination of Compound 1 and mouse PD-1 antibody is significantly more effective in inhibiting tumor growth than either drug alone. The results are summarized in Table 2.

TABLE 2
Results of in vivo tests of anti-tumor efficacy
of the compound in the B16F10 models

| Groups | Tumor Volume (mm³) | TGI (%) | p-Value |
|---|---|---|---|
| Vehicle Control | 1235.87 ± 220.28 | — | — |
| mPD-1 antibody, | 693.74 ± 272.23 | 43.87 | 0.0007 |
| 10 mg/kg, IP, BIW | | | |
| Compound 1, 30 mg/kg, | 795.99 ± 112.92 | 35.59 | 0.0098 |
| PO, BID | | | |
| Compound 1, 60 mg/kg, | 755.48 ± 155.85 | 38.87 | 0.0037 |
| PO, BID | | | |
| Compound 1, 120 mg/kg, | 650.67 ± 157.53 | 47.35 | 0.0002 |
| PO, BID | | | |
| mPD-1antibody, 10 | 487.05 ± 131.39 | 60.59 | <0.0001 |
| mg/kg, IP, BIW + | | | |
| Compound 1, 60 mg/kg, | | | |
| PO, BID | | | |

### Example 4: In Vivo Test of Anti-Tumor Efficacy of Compound 1 and Human PD-1 Antibodies in the 4T1 Humanized Mouse Model

4T1 tumor cells (ATCC, CRL-2539™) are maintained in vitro as a monolayer culture in RPMI 1640 medium supplemented with 10% heat inactivated fetal bovine serum at 37° C. in an atmosphere of 5% CO₂ in air. The tumor cells are routinely subcultured three times weekly by trypsin-EDTA treatment. Each mouse is inoculated subcutaneously at the fourth mammary pad with 1×10⁵ in 0.05 mL base medium for tumor development.

Compound 1 plus Opdivo (nivolumab): 40 BALB/c mice are inoculated subcutaneously at the fourth mammary pad with 4T1 cells for tumor development. On the six days of post inoculation, the mice are assigned into 4 groups using stratified randomization with 10 mice in each group based upon their tumor volume. The treatments are started from the day of randomization, and the groups receive the following treatments:

Group 1: Vehicle control;
Group 2: Opdivio, 10 mg/kg, i.p., BIW;
Group 3: Compound 1, 60 mg/kg, p.o., BID and
Group 4: Compound 1, 60 mg/kg, p.o., BID+Opdivio, 10 mg/kg, i.p., BIW respectively.

The tumor sizes are measured three times per week during the treatment.

The tumor growth inhibition effect of the combination of Compound 1 and Opdivo is examined. The tumor inhibition rate is calculated as:

TGI (%)=(1−(the volume of the tumor on the day of administration−the volume of the tumor on the first day of administration (treatment group))/(the volume of the tumor on the day of administration−the volume of the tumor on the first day (vehicle group)))×100%.

The results summarized in Table 3 show the tumor growth inhibition (%) of four groups at day 13. The tumor growth inhibition (%) of the combination group (Opdivo+Compound 1) is 34.65% at day 14, while the tumor growth inhibitions (%) of the Opdivo group and Compound 1 group are 21.88% and 21.70% respectively at day 13. The results show that the combination of Compound 1 and Opdivo is significantly more effective in inhibiting tumor growth than either drug alone at day 13. NB: Opdivo is a human antibody to human PD-1, and it has some toxicity in mice. After the 5th dose of Opdivo at day 14, mice are found dead within 1 hour of dosing. Thus, data are collected only up to day 13.

TABLE 3
Results of in vivo test of anti-tumor efficacy of the compound
and PD-1 antibody Opdivo in the 4T1 humanized mice model

| | TGI (%) | |
|---|---|---|
| Groups | at day 13 | p-Value |
| Vehicle Control | — | — |
| Opdivo(anti-hPD-1), | 21.88 | <0.0001 |
| 10 mg/kg, IP, BIW | | |
| Compound 1, 60 mg/kg, | 21.70 | <0.0001 |
| PO, BID | | |
| Compound 1, 60 mg/kg, PO, | 34.65 | <0.0001 |
| BID + Opdivo(anti-hPD-1), | | |
| 10 mg/kg, IP, BIW | | |

Compound 1 plus Keytruda (pembrolizumab): 40 BALB/c mice are inoculated subcutaneously at the fourth mammary pad with 4T1 cells for tumor development. On the six days of post inoculation, the mice were assigned into 4 groups using stratified randomization with 10 mice in each group based upon their tumor volume. The treatments were started from the day of randomization, and the groups receive the following treatments:

Group 1: Vehicle control;
Group 2: Keytruda, 10 mg/kg, i.p., BIW;
Group 3: Compound 1, 60 mg/kg, p.o., BID, and
Group 4: Compound 1, 60 mg/kg, p.o., BID+Keytruda, 10 mg/kg, i.p., BIW respectively.

The tumor sizes are measured three times per week during the treatment.

The tumor growth inhibition effect of the combination of Compound 1 and Keytruda is examined. The tumor inhibition rate is calculated as:

TGI (%)=(1−(the volume of the tumor on the day of administration−the volume of the tumor on the first day of administration (treatment group))/(the volume of the tumor on the day of administration−the volume of the tumor on the first day (vehicle group))×100%.

The results summarized in Table 4 shows the tumor growth inhibition (%) of four groups at day 10. The tumor growth inhibition (%) of the combination group (Keytruda+Compound 1) is 23.85% at day 10, while the tumor growth inhibitions (%) of the Keytruda group and Compound 1 group are 9.12% and 22.36% respectively at day 10. The results showed that the combination of Compound 1 and Keytruda is more effective in inhibiting tumor growth than either drug alone at day 10. NB: Keytruda is a humanized antibody to human PD-1, and it has some toxicity in mice. After the 4th dose of Keytruda at day 10, mice are found dead within 1 hour of dosing. Thus, data are collected before the dosing of Keytruda on day 10.

TABLE 4
Results of in vivo test of anti-tumor efficacy of the compound
and PD-1 antibody Keytruda in the 4T1 humanized mice model

| | TGI (%) | |
|---|---|---|
| Groups | at day 10 | p-Value |
| Vehicle Control | — | — |
| Keytruda (anti-hPD-1), | 9.12 | >0.5 |
| 10 mg/kg, IP, BIW | | |
| Compound 1, 60 mg/kg, | 22.36 | <0.0001 |
| PO, BID | | |
| Compound 1, 60 mg/kg, PO, | 23.85 | <0.0001 |
| BID + Keytruda (anti-hPD- | | |
| 1), 10 mg/kg, IP, BIW | | |

While the present invention has been described with reference to embodiments, it will be understood by those skilled in the art that various modifications and variations may be made therein without departing from the scope of the present invention as defined by the appended claims.

1-21. (canceled)

22. A method for treating a cancer, comprising administering to a subject in need thereof a small molecule inhibitor of the PD-1/PD-L1 interaction or a pharmaceutically acceptable salt or prodrug thereof and an anti-PD-1 antibody, wherein the small molecule inhibitor is not protein.

23. The method as described in claim 22, wherein the small molecule inhibitor is an aromatic vinyl or aromatic ethyl derivative;
and/or, the small molecule inhibitor has a molecular weight (MW) of less than 1500 Daltons;
and/or, the small molecule inhibitor has an IC₅₀ of less than 100 nM in a PD-1/PD-L1 binding assay.

24. The method as described in claim 22, wherein the small molecule inhibitor binds to PD-L1.

25. The method as described in claim 22, wherein the small molecule inhibitor is a compound selected from the compounds identified in Method 1.8, 1.9, 1.10, or 1.11, supra, in free or pharmaceutically acceptable salt form.

26. The method as described in claim 22, wherein the small molecule inhibitor is 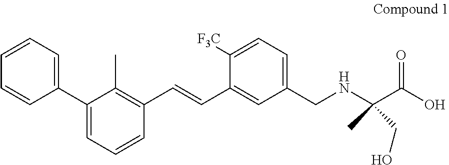 [SMILES: Cc1c(/C=C/c2cc(CN[C@@](C)(CO)C(=O)O)ccc2C(F)(F)F)cccc1-c1ccccc1]
in free or pharmaceutically acceptable salt form.

27. The method as described in claim 22, wherein the cancer is cervical carcinomas, renal cell carcinoma, melanomas, breast cancer, colorectal cancer, or head and neck squamous cell carcinoma (HNSCC).

28. The method as described in claim 22, wherein the cancer is breast cancer, melanomas or colorectal cancer.

29. The method as described in claim 22, wherein the small molecule inhibitor is administered orally.

30. The method as described in claim 22, wherein the small molecule inhibitor is administered at a total dose of 20-300 mg/kg or 30-240 mg/kg per day.

31. The method as described in claim 22, wherein the small molecule inhibitor is simultaneously administered with the antibody.

32. The method as described in claim 22, wherein the antibody is a monoclonal antibody.

33. The method as described in claim 22, wherein the antibody is pembrolizumab, nivolumab, cemiplimab, toripalimab, camrelizumab or sintilimab.

34. The method as described in claim 22, wherein the antibody is administered intravenously or subcutaneously.

35. The method as described in claim 22, wherein the antibody is administered at a dose of 0.1-50 mg/kg, 0.2-10 mg/kg, 0.3-5 mg/kg, 0.4-5 mg/kg, 0.5-5 mg/kg, 0.6-4 mg/kg, 0.6-3 mg/kg, 0.6-2 mg/kg, 0.8-4 mg/kg, 0.8-3 mg/kg, 0.8-2 mg/kg, 1-10 mg/kg, 1-5 mg/kg, 1-4 mg/kg, 1-3 mg/kg, 1-2 mg/kg or 2-3 mg/kg twice a week (BIW), once every week, once every two weeks, once every three weeks or once every four weeks.

36. The method as described in claim 22, wherein the subject has previously received cancer treatment and wherein the subject is not responsive to the previous cancer treatment.

37. The method as described in claim 36, wherein the previous cancer treatment is chemotherapy.

38. The method as described in claim 22, which further comprising administration of an additional anti-cancer agent.

39. The method as described in claim 37, wherein the chemotherapy comprises a platinum containing chemotherapeutic agent.

40. The method as described in claim 37, wherein the chemotherapy is platinum-containing doublet chemotherapy.
